(19) United States
(12) Patent Application Publication
(10) Pub. No.: US 2025/0248605 A1
(43) Pub. Date: Aug. 7, 2025

(54) SENSING AND DIAGNOSING ADVERSE HEALTH EVENT RISK

(71) Applicant: Medtronic, Inc., Minneapolis, MN (US)

(72) Inventors: Shantanu Sarkar, Roseville, MN (US); Val D. Eisele, III, Little Canada, MN (US); Jerry D. Reiland, Coon Rapids, MN (US); Steven G. Nelson, Raynham, MA (US); Jodi L. Redemske, South Haven, MN (US); Juliana E. Pronovici, New Hope, MN (US); Shubha Majumder, Minneapolis, MN (US); Gautham Rajagopal, Minneapolis, MN (US); Yong Cho, Excelsior, MN (US); Bruce D. Gunderson, Plymouth, MN (US); Andrew Radtke, Minneapolis, MN (US); John E. Burnes, Blaine, MN (US); Ryan D. Wyszynski, Oak Grove, MN (US); James Libbey, Minnetonka, MN (US)

(21) Appl. No.: 18/856,860

(22) PCT Filed: Apr. 13, 2023
(86) PCT No.: PCT/IB2023/053802

§ 371 Date: Oct. 14, 2024

Related U.S. Application Data
(60) Provisional application No. 63/363,440, filed on Apr. 22, 2022.

(51) Int. Cl. A61B 5/0205; A61B 5/00; A61B 5/024; A61B 5/11; A61B 5/352; A61B 5/366

(52) U.S. Cl. CPC A61B 5/0205; A61B 5/1118; A61B 5/352; A61B 5/366; A61B 5/4561; A61B 5/6847; A61B 5/02405

(57) ABSTRACT

An example system includes an implantable medical device and processing circuitry. The processing circuitry is configured to determine a first respective one or more values for each of first physiological parameters, the first physiological parameters including one or more subcutaneous tissue impedance parameters determined from one or more subcutaneous tissue impedance measurements, determine a second respective value for each of second physiological parameters, the second physiological parameters including one or more second parameters determined from one or more second measurements, identify a first diagnostic state for each of the first physiological parameters based on the first respective values, identify a second diagnostic state for each of the second physiological parameters based on the second respective values, the first and second diagnostic states defining inputs for a probability model, and determine, from the probability model, a probability score indicating a likelihood of an adverse health event.

This application claims the benefit of U.S. Provisional Application Ser. No. 63/363,440, filed Apr. 22, 2022, the entire content of which is incorporated herein by reference.

### FIELD

This disclosure generally relates to systems including medical devices and, more particularly, to monitoring of patient health using such systems.

### BACKGROUND

A variety of devices are configured to monitor physiological signals of a patient. Such devices include implantable or wearable medical devices, as well as a variety of wearable health or fitness tracking devices. The physiological signals sensed by such devices include as examples, electrocardiogram (ECG) signals, respiration signals, perfusion signals, activity and/or posture signals, pressure signals, blood oxygen saturation signals, body composition, and blood glucose or other blood constituent signals. In general, using these signals, such devices facilitate monitoring and evaluating patient health over a number of months or years, outside of a clinic setting. In some cases, some medical devices have been used or proposed for use to monitor heart failure (HF) or to detect HF events, such as heart failure decompensation or hospitalization, or other health events, such as sudden cardiac death (SCD).

HF is the most common cardiovascular disease that causes significant economic burden, morbidity, and mortality. In the United States alone, roughly 5 million people have HF, accounting for a significant number of hospitalizations. HF may result in cardiac chamber dilation, increased pulmonary blood volume, and fluid retention in the lungs. Acute decompensated HF is a manifestation of worsening HF or broadly chronic illness symptoms that requires HF admission to relieve patients of congestion and shortness of breath symptoms. Generally, the first indication that a physician has of worsening HF in a patient is not until it becomes a physical manifestation with swelling or breathing difficulties so overwhelming as to be noticed by the patient who then proceeds to be examined by a physician. This is undesirable since hospitalization at such a time would likely be required for a cardiac HF patient to remove excess fluid and relieve symptoms.

### SUMMARY

This disclosure describes techniques for providing an early warning for various health or heart conditions (e.g., HF decompensation, worsening HF, or other cardiovascular-related conditions, such as edema). The disclosed technology uses prediction and probability modeling to determine an indicator that an adverse health condition will occur or is occurring. In this manner, the disclosed techniques may allow detection or prediction of such events, e.g., even if there are no physical manifestations apparent. The probability or likelihood indication may include a probability score indication that provides a percentage or likeliness that a particular adverse health event will occur within a predetermined time period in the future (e.g., within the next 30 days or other desired period of time for knowing the likeliness).

The techniques of this disclosure may be implemented by systems including one or more IMDs and computing devices that can autonomously and continuously collect physiological parameter data while the IMD is implanted in a patient over months or years and perform numerous operations per second on the data to enable the systems herein to determine the health status of a patient. Using techniques of this disclosure with an IMD may be advantageous when a physician cannot be continuously present with the patient to evaluate the physiological parameters and/or where performing the operations on the data described herein could not practically be performed in the mind of a physician.

In some examples, the techniques and systems of this disclosure may use a machine learning model to more accurately infer the patient's condition, e.g., to risk of HF or another health event, based on physiological data collected by an IMD. In some examples, the machine learning model is trained with a set of training instances, where one or more of the training instances comprise data that indicate relationships between various sets of input data and outputs. Because the machine learning model is trained with potentially thousands or millions of training instances, the machine learning model may reduce the amount of error in risk level or other values useful for control of dialysis. Reducing errors using the techniques of this disclosure may provide one or more technical and clinical advantages, such as increasing the efficacy of therapies prescribed based on the output of the machine learning model.

In one example, a system comprises an implantable medical device (IMD) comprising a plurality of electrodes and configured for subcutaneous implantation in a patient, wherein the IMD is configured to determine one or more first measurements comprising subcutaneous tissue impedance measurements via the electrodes; and processing circuitry coupled to one or more storage devices, and configured to: obtain second measurements, the second measurements being different than the first measurements; determine a first respective one or more values for each of a plurality of first physiological parameters, wherein each of the plurality of first physiological parameters indicates a physiological status of the patient, the plurality of first physiological parameters determined from the one or more first measurements; determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from the one or more second measurements; identify a first diagnostic state for each of the first physiological parameters based on the first respective values and identify a second diagnostic state for each of the second physiological parameters based on the second respective values, the first and second diagnostic states defining a plurality of inputs for a probability model; and determine, from the probability model, a probability score indicating at least one of a likelihood that the patient (a) is experiencing an adverse health event of a chronic condition of the patient or (b) is to experience the adverse health event.

In one example, a method comprises determining a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements; determining a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements; identifying a first diagnostic state for each of the first physiological parameters based on the first respective values; identifying a second diagnostic state for each of the second physiological parameters based on the second respective values, the first and second diagnostic states defining a plurality of inputs for a probability model; and determining, from the probability model, a probability score indicating at least one of a likelihood that the patient (a) is experiencing an adverse health event of a chronic condition of the patient or (b) is to experience the adverse health event.

In one example, a non-transitory computer-readable storage medium comprising instructions that, when executed by processing circuitry, cause the processing circuitry to at least: determine a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements; determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements; identify a first diagnostic state for each of the first physiological parameters based on the first respective values; identify a second diagnostic state for each of the second physiological parameters based on the second respective values, the first and second diagnostic states defining a plurality of inputs for a probability model; and determine, from the probability model, a probability score indicating at least one of a likelihood that the patient (a) is experiencing an adverse health event of a chronic condition of the patient or (b) is to experience the adverse health event.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a block diagram illustrating an example system that includes medical device(s) used to obtain diagnostic states from the various physiological parameters.

FIG. 2 is a functional block diagram illustrating an example configuration of the external device of FIG. 1.

FIG. 3 illustrates a chart of heart rate with respect to activity intensity to determine a value of chronotropic competence/incompetence.

FIG. 4A is a chart showing an example of a 2-minute segment of sub-cutaneous impedance measurement done by IMD 10 at nighttime.

FIG. 4B is a chart showing an example of an envelope signal of the segment shown in FIG. 4A.

FIG. 4C is a chart showing an example of a phase plot of the envelope signal in FIG. 4B.

FIG. 5 is a block diagram illustrating an example of an AI model that may be used as an example of a probability model.

FIG. 6 is a block diagram illustrating an example of an ML model that may be used as an example of a probability model.

FIG. 7 illustrates an environment of an example medical system in conjunction with the patient, including an example implantable medical device (IMD) used to determine physiological parameters of the patient.

FIG. 8 is a functional block diagram illustrating an example configuration of an IMD of FIG. 7.

FIG. 9 is a flow diagram illustrating an example method that may be performed by one or more medical devices (e.g., IMDs) and/or a computing device to determine a probability score with respect to the patient, in accordance with one or more techniques disclosed herein.

FIG. 10 is a flow diagram illustrating an example method that may be performed by one or both the medical device(s) and external device shown in FIG. 1 to provide instructions with respect to probability score, in accordance with one or more techniques disclosed herein.

FIG. 11 is a conceptual diagram illustrating an example ML model configured to determine a risk level of a health event based on physiological parameter values of a patient.

FIG. 12 is a conceptual diagram illustrating an example training process for a ML model, in accordance with examples of the current disclosure.

FIG. 13A is a perspective drawing illustrating an example IMD.

FIG. 13B is a perspective drawing illustrating another example IMD.

Like reference characters refer to like elements throughout the figures and description.

### DETAILED DESCRIPTION

This disclosure describes techniques for providing an early warning for various health or heart conditions using prediction and probability modeling to determine a probability or likeliness indicator that an adverse health condition will occur or is occurring. In some examples, the probability score may be based on respective physiological parameter values corresponding to physiological parameters acquired from one or more medical devices. Processing circuitry of a device, e.g., a remote server, tablet, smartphone, or one or more implanted, patient-worn, or external medical devices (which may have sensed values of one or more of the physiological parameters) may determine respective values for each physiological parameter, and determine the probability score based on the physiological parameter values.

In some examples, the prediction and/or probability modeling according to the techniques described herein may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers, deep learning models), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model-selection techniques, such as Bayesian information criterion (BIC) or Akaike information criterion (AIC), may be used to evaluate probability models prior to use.

Implantable medical devices (IMDs) may sense and monitor ECGs and other physiological signals, and monitor physiological parameters. Example IMDs include pacemakers and implantable cardioverter-defibrillators, which may be coupled to intravascular or extravascular leads, as well as pacemakers with housings configured for implantation within the heart, which may be leadless. Some IMDs do not provide therapy, such as implantable patient monitors. One example of such an IMD is the Reveal LINQ™ or LINQ II™ Insertable Cardiac Monitor (ICM), available from Medtronic, Inc., which may be inserted subcutaneously. Such IMDs may facilitate relatively longer-term monitoring of patients during normal daily activities, and may periodically transmit collected data, e.g., episode data for detected arrhythmia episodes, to a remote patient monitoring system, such as the Medtronic Carelink™ Network.

In some of the following examples, techniques described in U.S. application Ser. No. 16/450,250 by Sarkar et al., entitled “SENSING RESPIRATION PARAMETERS BASED ON AN IMPEDANCE SIGNAL,” filed on Jun. 24, 2019, U.S. application Ser. No. 17/021,489 by Sarkar et al., entitled “DETERMINING HEART CONDITION STATUSES USING SUBCUTANEOUS IMPEDANCE MEASUREMENTS” filed on Sep. 15, 2020, and U.S. application Ser. No. 17/021,564 by Sarkar et al., “DETERMINING HEALTH CONDITION STATUSES USING SUBCUTANEOUS IMPEDANCE MEASUREMENTS” filed Sep. 15, 2020, are each incorporated herein by reference in their entirety.

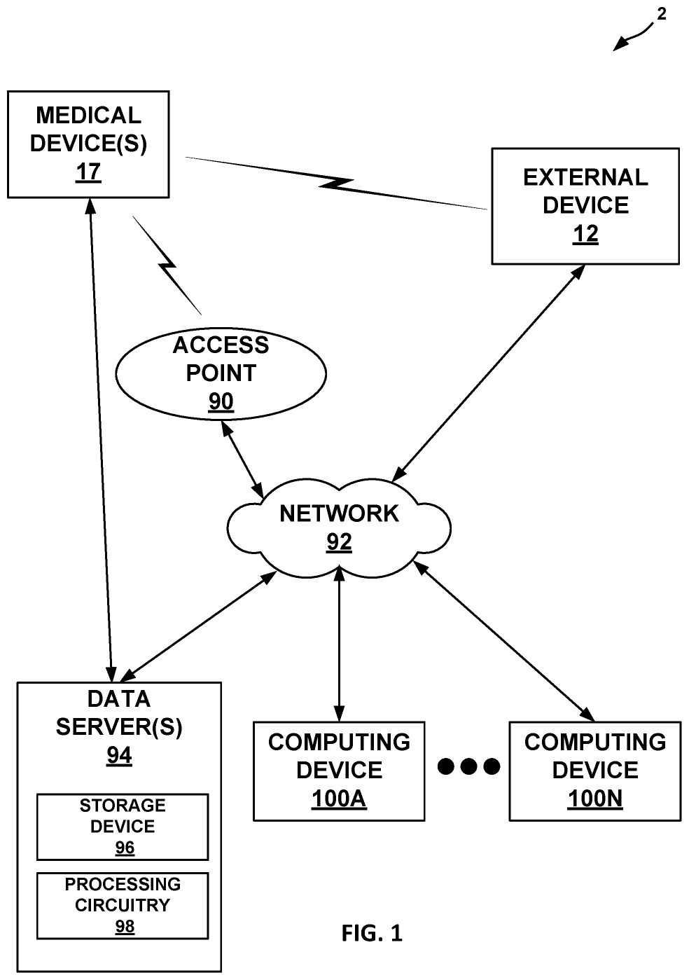

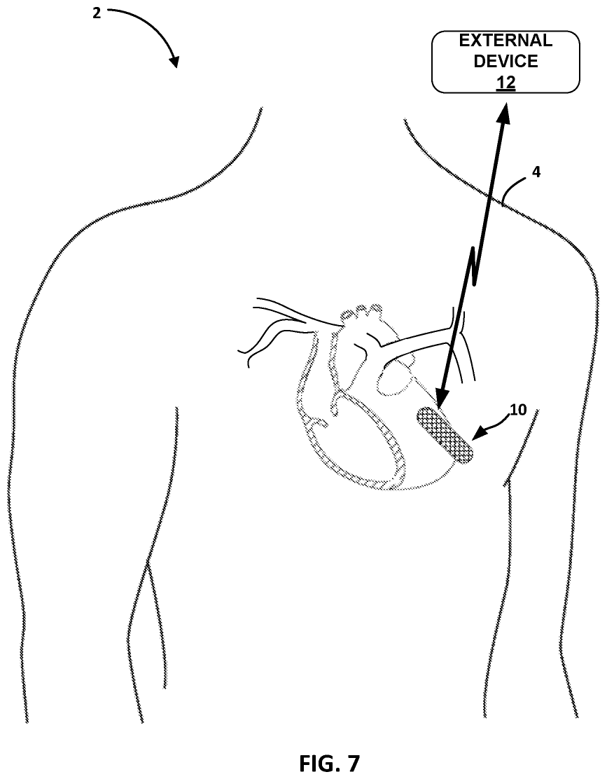

FIG. 1 is a block diagram illustrating an example system 2 that includes one or more medical device(s) 17, an access point 90, a network 92, external computing devices, such as data servers 94, and one or more other computing devices 100A-100N (collectively, “computing devices 100”). In some examples, medical device(s) 17 may include an implantable medical device (IMD), such as IMD 10 described with reference to FIGS. 7-8, 13A and 13B. In this example, medical device(s) 17 may use communication circuitry 54 to communicate with external device 12 via a first wireless connection, and to communicate with an access point 90 via a second wireless connection.

In one or more of the various example techniques described with reference to FIG. 1, access point 90, external device 12, data server(s) 94, and computing devices 100 may be interconnected and may communicate with each other through network 92. Network 92 may include a local area network, wide area network, or global network, such as the Internet. The example system described with reference to FIG. 1 may be implemented, in some aspects, with general network technology and functionality similar to that provided by the Medtronic CareLink® Network, developed by Medtronic, Inc., of Minneapolis, MN.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient.

Medical device(s) 17 may be configured to transmit data, such as sensed, measured, and/or determined values of physiological parameters (e.g., heart rates, impedance measurements, impedance scores, fluid indices, respiratory rate, activity data, cardiac electrograms (ECGs), historical physiological data, blood pressure values, posture, chronotropic incompetence, short-term heart rate variability, sleep disordered breathing, R-wave morphology, etc.), to access point 90 and/or external device 12. In some examples, medical device(s) 17 may be configured to determine multiple physiological parameters. In some examples, medical device(s) 17 may include an IMD 10 configured to determine respiration rate values, subcutaneous tissue impedance values, and ECG values. In such examples, IMD 10 may provide multiple physiological parameters. In some examples, medical device(s) 17 may include one or more of bed sensors to monitor parameters such as sleep respiration, weight scales to monitor parameters such as weight, pulse oximeter to monitor parameters such as oxygenation, radar based external sleep apnea devices, infrared cameras to monitor parameters such as jugular venous distension, smartphone application to track parameters such as voice abnormalities, smart device to receive inputs from a user, such as the patient, to indicate patient is not feeling well or is feeling panicked and/or is having a panic attack, and continuous glucose monitor to monitor blood glucose. In some examples, medical device(s) 17 may include biochemical sensors to determine parameters such as an abnormal glucose level of other blood analytes, such as cortisol, adrenaline, etc., that may indicate a patient is panicked and/or is having a panic attack. In some examples, medical device(s) 17 may include a wearable computing device that may include electrodes and other sensors to sense physiological parameters and may collect and store physiological data and detect episodes based on such parameters. The wearable computing device may be incorporated into the apparel of patient, such as within clothing, shoes, eyeglasses, a watch or wristband, a hat, etc. In some examples, wearable computing device is a smartwatch or other accessory or peripheral for a smartphone computing device. Access point 90 and/or external device 12 may then communicate the retrieved data to data server(s) 94 via network 92.

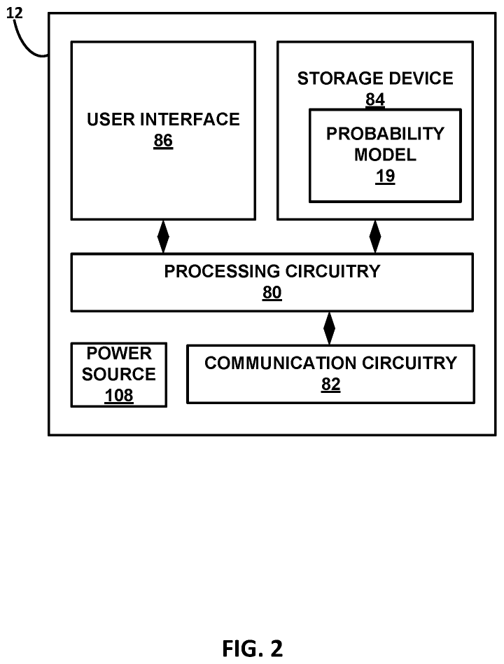

In some instances, one or more of medical device(s) 17 may transmit data over a wired or wireless connection to data server(s) 94 or to external device 12. For example, data server(s) 94 may receive data from medical device(s) 17 or from external device 12. In another example, external device 12 may receive data from data server(s) 94 or from medical device(s) 17, such as physiological parameter values, diagnostic states, or probability scores, via network 92. In such examples, external device 12 may determine the data received from data server(s) 94 or from medical device(s) 17 and may store the data to storage device 84 (FIG. 2) accordingly.

In addition, one or more of medical device(s) 17 may serve as or include data server(s) 94. For example, medical device(s) 17 may include enough storage capacity or processing power to perform the techniques disclosed herein on a single one of medical device(s) 17 or on a network of medical device(s) 17 coordinating tasks via network 92 (e.g., over a private or closed network). In some examples, one of medical device(s) 17 may include at least one of the data server(s) 94. For example, a portable/bedside patient monitor may be able to serve as a data server, as well as serving as one of medical device(s) 17 configured to obtain physiological parameter values from patient 4. In other examples, data server(s) 94 may communicate with each of medical device(s) 17, via a wired or wireless connection, to receive physiological parameter values or diagnostic states from medical device(s) 17. In a non-limiting example, physiological parameter values may be transferred from medical device(s) 17 to data server(s) 94 and/or to external device 12.

As shown in FIGS. 1, 2, and 8, processing circuitry 50, 80, 98 is located in a respective IMD 10, external device 12, and data server(s) 94. For simplicity, system 2 will be referenced in the examples discussed below, but any one or more of processing circuitries 50, 80, and 98 may be perform one or more of functions performed by system 2 discussed below. As shown in FIGS. 2, 3, and 5, communication circuitry 60, 140, 24 is located in a respective IMD 10, patient computing devices 12, and computing systems 20. For simplicity, communication circuitry 60 will be referenced in the examples discussed below, but any one or more of communication circuitries 60, 140, and 24 may be used as the processing circuitry.

In some examples, system 2 may use physiological parameters from one or more of three physiological parameter categories to determine a probability score indicating a likelihood that the patient is experiencing an adverse health event or is likely to experience the adverse health event within a predetermined amount of time. For example, the physiological parameters categories may include a physiological status, a precipitating condition, and a change in symptoms and/or functional capacity. Some examples of a physiological status include heart rate, respiratory rate, respiratory effort, fluid status, sympathetic tone, HRV, blood pressure, weight change, fluid redistribution, tissue perfusion, pulse oxygenation, sleep disordered breathing, heart sounds, and ECG QRST morphology (R-wave amplitude, slope, width). Some examples of a precipitating condition include current clinical status, clinical history, alternate medical problem like pneumonia, sepsis, respiratory infection, chronic obstructive pulmonary disease (COPD), one or more sleep apnea events occurring at night, atrial fibrillation (AF) with rapid ventricular rate (RVR), anemia, physical exertions, hypoxemia, hyperglycemia, hypoglycemia, increased A1C levels, panic attack, dietary change, dietary compliance, medication change, and medication compliance, reduction in urinary output, increased fluid consumption, sleep apnea, Cheyenes Stokes breathing, sleep apnea burden, and premature ventricular contractions (PVC) burden. Some examples of symptoms that may change may include respiratory rate, respiratory effort, rales through lung sounds, symptom app, coughing, cough frequency, chronotropic incompetence, hematocrit, and peripheral perfusion of incident infection. Some examples of functional capacity that may change may include activity, voice pattern, sleep posture, gait, speech pattern, and sit-to-stand time, which is the period of time it takes a person to move from a sitting position to a standing position. In some examples, system 2 may receive information on one or more of the above parameters and/or additional parameters not listed above from an electronic medical record (EMR) system.

In some examples, providing different types of physiological parameters, such as from different physiological parameter categories, such as physiological status, precipitating condition, change in symptoms, and a change in functional capacity, to the probability model may help improve the efficiency and accuracy of determining a probability score. However, certain types of physiological parameters may provide a stronger indication for determining an adverse health condition and may be more heavily weighted in the probability model to determine a probability score indicating a likelihood that the patient is experiencing an adverse health event or is likely to experience the adverse health event within a predetermined amount of time.

Some types of physiological parameters, by themselves, may not provide accurate determinations of an adverse health condition. However, those physiological parameters, when combined with one or more other physiological parameters, may improve the accuracy in determining an adverse health condition. Accordingly, inputting multiple different physiological parameters may improve the accuracy of a probability score indicating a likelihood that the patient is experiencing an adverse health event or is likely to experience the adverse health event within a predetermined amount of time.

In some examples, processing circuitry may determine a measure of chronotropic incompetence to determine an adverse health event. Heart rate is a basic compensatory mechanism in HF. When heart starts failing, or patient develops acute decompensated HF, the patient responds by increasing heart rate to compensate for decreasing stroke volume to maintain cardiac output. The heart rate response depends on the heart rate reserve a patient has. Similar to a compensatory mechanism in HF, heart rate may also increase with activity. The amount of increase may determine how chronotropic competent or incompetent the patient is and also determines an amount of heart rate reserve the patient has. Thus, a patient with HF or developing HF, specifically with preserved ejection fraction (EF), may have less heart rate reserve (i.e. more chronotropic incompetence), may not be able to compensate very well and may develop symptoms which may require hospitalization. Thus, a chronotropic incompetent patient may be more likely to be hospitalized for HF.

In some examples, medical device(s) 17, such as IMD 10, may measure chronotropic incompetence by making high-resolution measurement of heart rate and activity simultaneously. In some examples, medical devices(s) 17 may measure activity intensity (or activity count), such as by using an accelerometer, for a period of time such as every 5 minutes. High-resolution measurements are measured at a greater rate than low-resolution measurements. In some examples, high-resolution measurements may be made every 5 minutes, every 10 minutes, or any other similar time period. In some examples, low-resolution measurements may be made every day, every week, or every month. While high-resolution measurements may require more power and storage space over a period of time than low-resolution measurements, high-resolution measurement may provide greater details on a parameter being measured. Medical device(s) may also measure the average heart rate over the same period of time such as every 5 minutes.

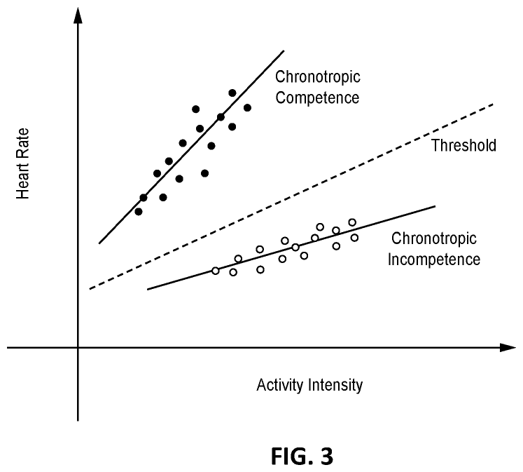

FIG. 3 illustrates an example graph of heart rate values plotted with respect to activity intensity. FIG. 3 also illustrates a threshold for determining whether particular activity and heart rate values may label a patient as chronotropic incompetent or chronotropic competent. To determine a chronotropic incompetence threshold, system 2 may collect the measurements over a period of time, such as a 24-hour period, from one or more patients, and apply line fits to determine clusters of chronotropic incompetence and competence. System 2 may also determine quantify a rate of change of heart rate as a function of change in activity over the period of time (e.g., 24-hour period) as a threshold between chronotropic competence and incompetence. System 2 may measure a value of chronotropic incompetence/competence using the slope of the fitted line.

In addition, system 2 may use other parameters, such as goodness of fit, or the intercept to improve the robustness of the chronotropic incompetence measurement. In some examples, system 2 may collate these high-resolution measurements over different periods of time (e.g., 24 hours, 7 days, 30 days, etc.). In some examples, system 2 may estimate the chronotropic incompetent parameter data for every day, and then features such as 7-day or 30-day average or minimum or maximum or coefficient of variation may be computed. In addition, a number of days a measurement is above or below a threshold over a period of time may also be used as a metric. In some examples, system 2 may compare these individual metrics against a threshold to determine whether patient has chronotropic incompetence and/or a value of the chronotropic incompetence. In some examples, system 2 may combine multiple metrics using decision trees or fuzzy logic or a risk score using multivariate logistic regression or machine learning techniques. Further, chronotropic incompetence may be used as a static parameter in a prediction model. In some examples, chronotropic incompetence may not be computed during AF.

As an example, shown in an example in Table 1 below, an initial development set included 42 patients from an IMD-HF study who had at least 75 days of follow-up to include a diagnostic evaluation period and a clinical evaluation period with 15 days of blanking post implant. Thus, the first 15 days post implant are not included in the evaluation. There was a total of 225 monthly evaluations and 20 months (in 14 patients) with HF events resulting in an overall HF event rate of 8.9% of monthly evaluations. The results for 30-day evaluation segments where the chronotropic incompetence measurements could be performed is shown in Table 1. The metric used in Table 1 is a MaxSlope in the past 30 days being greater than 35% or less than or equal to 35%. As shown in Table 1, patients whose 30-day evaluations have a maximum slope less than or equal to 35% are 1.5 times more likely to be admitted for HF rather than patients with maximum slopes greater than 35%.

TABLE 1

| | Number | Number of | |
|---|---|---|---|
| | monthly | events | |
| MaxSlope = maximum | evaluations | admitted | Ratio of |
| daily slope in last 30 days | (%) | for HF (%) | events |
| MaxSlope >35% | 48 (28%) | 3 (6.3%) | Reference |
| MaxModeSum ≤35% | 126 (72%) | 12 (9.5%) | 1.52 |

Accordingly, as shown above, system 2 may use a physiological value, such as heart rate, in conjunction with a functional capacity value, such as activity intensity, to determine a value of chronotropic competence/incompetence, to determine whether a patient has an increased chance to be admitted for HF (or of another adverse health event).

In some examples, the determined value of chronotropic competence/incompetence may be combined with subcutaneous tissue impedance values to determine a probability score indicating a likelihood that the patient is experiencing an adverse health event or is likely to experience the adverse health event within a predetermined amount of time. In some examples, system may use additional parameters to indicate a confounding event occurred, such as a patient being in a wheelchair, or a patient falling and adjust the probability score based on the confounding event to indicate a likelihood that the patient is experiencing an adverse health event or is likely to experience the adverse health event. In some examples, in response to system 2 may notify a clinician to further investigate the confounding event.

In some examples, medical device(s) 17 may measure posture (e.g., body angle) to determine an adverse health event. In non-HF patients, body posture may be close to horizontal during sleep time and close to upright during active periods, such as during daytime. In HF patients, patients may have fluid overload and feel symptomatic leading to different postures of sleeping (e.g., with larger number of pillows) to make it easier to breathe. Further, daytime active posture may not be upright due to factors such as, but not limited to, illness and HF.

Medical device(s) 17, such as IMD 10, may make measurements, such as using a 3-axis accelerometer, to determine body posture during sleep. In some examples, medical device(s) may include one or more external devices, such as a smartphone, tablet, and Internet of Things (IoT) devices, to determine body posture with various sensors such as cameras, accelerometers, gyroscopes, etc. IoT devices may include, as examples, so called “smart” speakers, cameras, televisions, lights, locks, thermostats, appliances, actuators, controllers, or any other smart home (or building) devices. An upright reference may be derived by finding active times during daytime and taking an aggregate of the body posture during those times as an upright posture. As an example, when patient is active may be determined by identifying activity count average measurements which are greater than median activity count average during day+0.8*(maximum activity count average during day-median activity count average during day. Activity count average may be computed as the average of activity count every minute over a period of time, such as a 5-minute period. In some examples, the median of the measurements during above defined active periods may be used as an upright reference.

In some examples, system 2 may determine activity during rest time by determining when activity count average during nighttime hours is less than the median activity count average during nighttime hours. System 2 may determine a median posture as an angle between an x,y,z measurement against a fixed reference and obtaining a median of the angles or by obtaining a median of each x, y, and z measurements.

After a posture during maximum activity and a posture during rest at night is obtained, system 2 may obtain an angle between the two postures by determining a dot product between the two postures (e.g., x,y,z values from accelerometer). This angle is a metric of the posture difference. When patient is doing well and is not in HF, this angle is expected to be close to 90 degrees. If patient is not doing well, such as experiencing HF, this angle will drop to values below 90 degrees.

In some examples, system 2 may estimate an angle parameter for every day, and then determine features such as 7-day or 30-day average, minimum, maximum or coefficient of variation as well as number of days above a certain angle in the last 7 or 30 days. In some examples, system 2 may compare these individual metrics against a respective threshold to determine whether patient has reduced/increased difference in daytime/active posture versus nighttime rest posture. System 2 may combine multiple metrics using decision trees or fuzzy logic or a risk score using multivariate regression or machine learning techniques.

As an example, as shown in an example in Table 2 below, an initial development set included 42 patients from an IMD-HF study who had at least 75 days of follow-up to include a diagnostic evaluation period and a clinical evaluation period with 15 days of blanking post implant. There was a total of 225 monthly evaluations and 20 months (in 14 patients) with HF events resulting in an overall HF event rate of 8.9% of monthly evaluations. The results for 30-day evaluation segments where posture difference measurements may be performed is shown in Table 2 for a metric of patients which have at least one day with posture difference angle ≥90 degrees and patients with zero days with posture difference angle ≥90 degrees.

TABLE 2

| | Number | | |
|---|---|---|---|
| MaxSlope = maximum | monthly | Number of | Ratio of |
| slope in last 30 days | evaluations (%) | events (%) | events |
| Angle ≥90° on ≥1 of last 30 | 85 (38%) | 5 (5.9%) | Reference |
| days | | | |
| Angle ≥90° on 0 of last 30 | 140 (62%) | 15 (10.7%) | 1.8 |
| days | | | |

Patients with 30-day evaluations which have at least one day with posture difference angle ≥90 degrees were 1.8 times less likely to be admitted for HF rather than patient with lower posture differences. Accordingly, as shown above, system 2 may use measurements that determine activity in conjunction with other measurements to determine a posture difference angle over a period of time to determine whether a patient has an increased chance to be admitted for HF (or of another adverse health event).

In some examples, the determined posture difference angle over a period of time may be combined with subcutaneous tissue impedance values to determine a probability score indicating a likelihood that the patient is experiencing an adverse health event or is likely to experience the adverse health event within a predetermined amount of time. In some examples, the determined posture difference angle over a period of time may be combined with various other physiological parameter values, such as, but not limited to, heart sounds or QRST morphology, to determine a probability score indicating a likelihood that the patient is experiencing an adverse health event or is likely to experience the adverse health event within a predetermined amount of time.

In some examples, medical device(s) 17 may measure short-term HRV, e.g., based on ECG data sensed by the medical device(s), to determine an adverse health event. Short-term HRV may be a surrogate measure for changes in autonomic tone, specifically increases in sympathetic tone, over a short period of time that patients have as a compensatory mechanism in response to worsening HF (e.g., acute decompensated HF). In patients not suffering from HF, there is a balance between sympathetic and parasympathetic tone and HRV is relatively high. In HF patients, a critical compensatory mechanism to a trigger that worsens hemodynamic status is to increase sympathetic tone, which in turn signals the kidneys to retain more fluid as well as to increase heart rate and increase vasoconstriction to preserve pressure. Increase in sympathetic tone may be manifested as reduced HRV and patients with reduced HRV may be more likely to develop HF symptoms, like shortness of breath, and may require hospitalizations to relieve symptoms.

Conventionally short-term HRV may be measured as the standard deviation of RR intervals (SDNN) over a short period of time, such as 5 minutes. In some examples, medical device(s) 17 may measure and store higher resolution heart rate histograms, such as every 5 minutes. In some examples, system 2 may use the histogram to compute various short-term HRV metrics, such as entropy or sparseness of distribution using a Kolmogorov-Smirnov (KS) test or mode-sum, determined as the ratio of the total num of RR intervals in two highest bins and the total number of points in the histogram.

In an example, in an IMD-HF study shown below in Table 3, a 5-minute RR interval mode-sum is computed and stored over the 5-minute period. The histogram bin size is 10 milliseconds (ms) and the extent of the histogram goes from 400 ms to 1200 ms, with values below or above those limits collapsed into the edge bins. When HRV is high, mode-sum value is low. When HRV is low, mode-sum value is high. In some examples, nightly HRV trends may be measured to indicate sleep disordered breathing or frequent limb movements which may lead to disrupted sleep. These high-resolution measurements are collated over a period (example 24 hours, 7 days, 30 days, etc.). System 2 may estimate the mode-sum parameter data for every day, and then determine features such as 7-day or 30-day average or minimum or maximum or coefficient of variation. Further, number of days above or below a certain threshold over a period may also be used as a metric. These individual metrics may be compared against a respective threshold to determine whether patient has low HRV. System 2 may combine multiple metrics using decision trees or fuzzy logic or a risk score using multivariate regression or machine learning techniques. In patients with AF, system 2 may detect AF, e.g., based on ECG data, and may not determine short-term HRV related metrics during periods of AF.

In the example shown in Table 3 below, the initial development set included 42 patients from the IMD-HF study who had at least 75 days of follow-up to include a diagnostic evaluation period and a clinical evaluation period with 15 days of blanking post implant. There was a total of 225 monthly evaluations and 20 months (in 14 patients) with HF events resulting in an overall HF event rate of 8.9% of monthly evaluations. The results for 30-day evaluation segments where the mode-sum measurements are performed is shown for a metric of patients 30-day evaluations which have maximum mode-sum greater than 50%.

TABLE 3

| MaxModeSum = maximum | Number | | |
|---|---|---|---|
| daily mode-sum in last 30 | monthly | Number of | Ratio of |
| days | evaluations (%) | events (%) | events |
| MaxModeSum ≤50% | 100 (45%) | 4 (4.0%) | Reference |
| MaxModeSum >50% | 125 (55%) | 16 (12.8%) | 3.2 |

Patients with 30-day evaluations which have a maximum mode-sum greater than 50%. were 3.2 times more likely to be admitted for HF than patients with a maximum mode-sum less than or equal to 50%. Accordingly, as shown above, system 2 may use measurements to determine short-term HRV to determine a mode-sum data over a period of time to determine whether a patient has an increased chance to be admitted for HF (or of another adverse health event).

In some examples, the determined mode-sum data over a period of time may be combined with subcutaneous tissue impedance values to determine a probability score indicating a likelihood that the patient is experiencing an adverse health event or is likely to experience the adverse health event within a predetermined amount of time. In some examples, the determined mode-sum data over a period of time may be combined with various other physiological parameter values, such as, but not limited to, heart sounds or QRST morphology, to determine a probability score indicating a likelihood that the patient is experiencing an adverse health event or is likely to experience the adverse health event within a predetermined amount of time.

Compensatory mechanisms activate in response to change in oxygen demand. One such situation is sleep disordered breathing in which the patient goes through period of none, or shallow breathing followed by periods of deeper breaths. A similar mechanism takes place in case of sleep apnea. Sleep disordered breathing or Cheyenne Stokes breathing are observed in patients with severe HF. These patients are more likely to be hospitalized for HF and have a lower survival rate. Measuring occurrences of sleep disordered breathing continuously may prove as a marker for increased risk for impending worsening HF or development of arrhythmias such as AF. In some examples, measuring occurrences of sleep disordered breathing may indicate an AF burden. In some examples, measuring occurrences of sleep disordered breathing may indicate a PVC burden. In some examples, medical device(s) 17 may monitor sleep disordered breathing or Cheyenne Stokes breathing to determine an adverse health event.

In some examples, medical device(s) 17, such as IMD 10, may measure interstitial impedance which has the capability of measuring changes in venous return from the tissue surrounding the electrodes due to changes in intrathoracic pressure during the inspiration and expiration cycle. IMD 10 may collate the measured interstitial impedance over a first period of time.

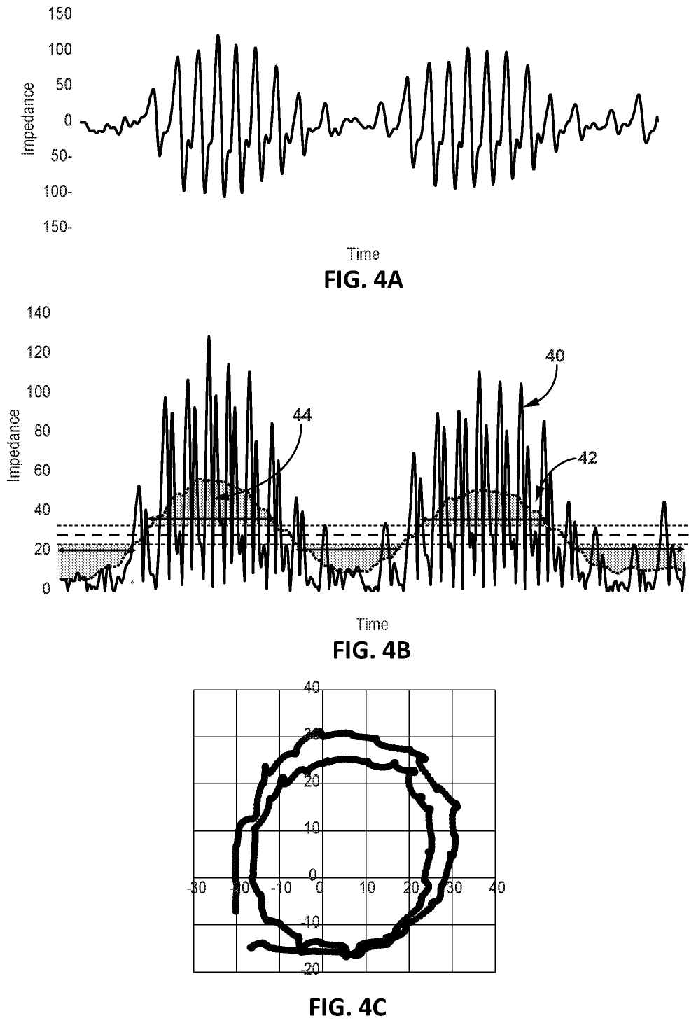

FIG. 4A shows a 2-minute segment of sub-cutaneous impedance measurement done by IMD 10 at nighttime. In some examples, IMD 10 takes the form of the Reveal LINQ™ or LINQ II™ ICM. In some examples, these measurements may be made by other implantable devices such as IPG, ICD and CRTD device using intra-cardiac electrodes or other subcutaneous devices like extra-vascular ICDs, or subcutaneous devices like patches.

FIG. 4B shows an example of system 2 measuring a degree of sleep disordered breathing by monitoring an envelope of the waveform and determine an intensity of change over time. In some examples, processing circuitry of system 2 subtracts a moving average from the signal to subtract low frequency trends. The processing circuitry may determine an absolute value 40, and apply a moving average (or low pass) filter to absolute value 40 to derive the envelope signal 42.

System 2 may use envelope signal to determine various features, such as taking the median of the envelope signal and computing the cumulative difference of the envelope signal from the +/−10% of the median, as shown in FIG. 4B. The shaded area 44 indicates how much the envelope is out of a normal range. As an example, if a patient is breathing normally this area 44 will be low or close to zero. In sleep disordered breathing, this area 44 will be high. Similarly, system 2 may use also use other metrics such as duration out of range (i.e. above or below +/−10% of median).

FIG. 4C shows an example for a phase plot for the signal above. System 2 may use a phase plot of the envelope signal at the expected cycle length to determine the presence of periodicity which is a hallmark of sleep disordered breathing. The example phase plot for the signal above is shown below. Impedance measurements may be corrupted by motion of a patient. Accordingly, sleep disordered breathing may preferably be computed when patient is not moving. Medical device(s) 17, such as a medical device 17 with a 3-axis accelerometer may be used to identify times when a patient is in motion (e.g. patient is active) and determine to use measurements segments where patients had no motion (e.g. patient is inactive) to determine sleep disordered breathing. In addition, large fluctuations in impedance signals indicating artifacts may also be detected and used to discriminate good signals from bad signals before sleep disordered breathing metrics are computed.

In an example IMD-HF study, an IMD-HF development set data included 42 patients from the IMD-HF study who had at least 75 days of follow-up to include a diagnostic evaluation period and a clinical evaluation period with 15 days of blanking post implant. There was a total of 225 monthly evaluations and 20 months (in 14 patients) with HF events resulting in an overall HF event rate of 8.9% of monthly evaluations. In this data cohort, five patients exhibited sleep disordered breathing patterns in the impedance signal and four of those patients (80%) were hospitalized for HF. Ten of the remaining 38 patients (26%) were hospitalized for HF but did not exhibit sleep disordered breathing.

Thus, patients with sleep disordered breathing showed a significantly greater likelihood to be hospitalized for HF. Accordingly, as shown above, system 2 may use interstitial impedance and activity measurements to determine sleep disordered breathing patterns to determine whether a patient has an increased chance to be admitted for HF (e.g. an adverse health event). In some examples, sleep disordered breathing may also be detected from ECG data, such as identifying a cyclical pattern of heart rate changes that is associated with sleep disordered breathing.

In some examples, the determined sleep disordered breathing patterns may be combined with subcutaneous tissue impedance values to determine a probability score indicating a likelihood that the patient is experiencing an adverse health event or is likely to experience the adverse health event within a predetermined amount of time.

Changes in R-wave morphology due to increased blood volume that happened during decompensated HF may be attributed to dilation of the ventricle, slowing of the R-wave conduction and change in vector of R-wave propagation due to increased volume. In normal compensated HF patients, a narrow complex R-wave may be present and with fluid retention and increased blood volume in the chambers of the heart the R-wave morphology widens, and the R-wave amplitude reduces.

In some examples, medical device(s) 17 may determine various parameters related to the R-wave morphology measured using a near field or far field ECG signal. R-wave amplitude, R-wave width as measured by the difference in positive to negative slew, maximum slew of R-wave, or the overall area under the R-wave may all be used as features. In some examples, a reduced R-wave amplitude or wider QRS complex may indicate increased fluid volume returning to the heart or increase in preload which is a common occurrence in acute decompensated HF.

System 2 may use deep learning networks such as convolution neural networks to automatically identify features. System 2 may estimate the R-wave morphology related parameters data for every day, and then determine features such as 7-day or 30-day average or minimum or maximum or coefficient of variation. Further, system 2 may also use a number of days above or below a respective threshold over a period of time as a metric. These individual metrics may be compared against threshold to determine whether patient is more likely to have lower R-wave amplitude or wider QRS complex. In general, with development of fluid the R-wave amplitude will decrease and QRS complex may widen over time. The relative decrease or increase in these parameters may be quantified using features such as cumulative difference of the long-term average and the short-term average over a finite period of time. System 2 may determine a patient has HF when this cumulative difference exceeds a threshold. In some examples, system 2 may combine multiple metrics using decision trees or fuzzy logic or a risk score using multivariate regression or deep learning neural networks techniques.

In the example shown in Table 4 below, the initial development set included 42 patients from an IMD-HF study who had at least 75 days of follow-up to include a diagnostic evaluation period and a clinical evaluation period with 15 days of blanking post implant. There was a total of 225 monthly evaluations and 20 months (in 14 patients) with HF events resulting in an overall HF event rate of 8.9% of monthly evaluations. The results for 30-day evaluation segments where the mode-sum measurements could be performed is shown in Table 4 below.

TABLE 4

| Cumsum2Max = ratio | | | |
|---|---|---|---|
| of cum-sum to maximum | Number | | |
| R-wave amplitude | monthly | Number of | Ratio of |
| in last 30 days | evaluations (%) | events (%) | events |
| Cum-sum2Max ≤80 | 117 (59%) | 5 (2.9%) | Reference |
| Cum-sum2Max >80 | 81 (41%) | 10 (12.3%) | 2.9 |

Patients with a ratio of a cumulative sum of R-wave amplitude to R-wave amplitude maximum over 30 days greater than 80% were 2.9 times more likely to be admitted for HF rather than patient with lower maximum mode-sum. Accordingly, as shown above, system 2 may use measurements to determine R-wave amplitudes over a period of time to determine whether a patient has an increased chance to be admitted for HF (or of another adverse health event).

In some examples, the determined ratio of a cumulative sum to maximum R-wave amplitude over 30 days may be combined with subcutaneous tissue impedance values to determine a probability score indicating a likelihood that the patient is experiencing an adverse health event or is likely to experience the adverse health event within a predetermined amount of time.

In some examples, two or more determined physiological parameters, such as the examples discussed above, may be combined with cardiac values to determine a probability score indicating a likelihood that the patient is experiencing an adverse health event or is likely to experience the adverse health event within a predetermined amount of time.

In some examples, providing more determined physiological parameters may improve the accuracy of a probability score indicating a likelihood that the patient is experiencing an adverse health event or is likely to experience the adverse health event within a predetermined amount of time.

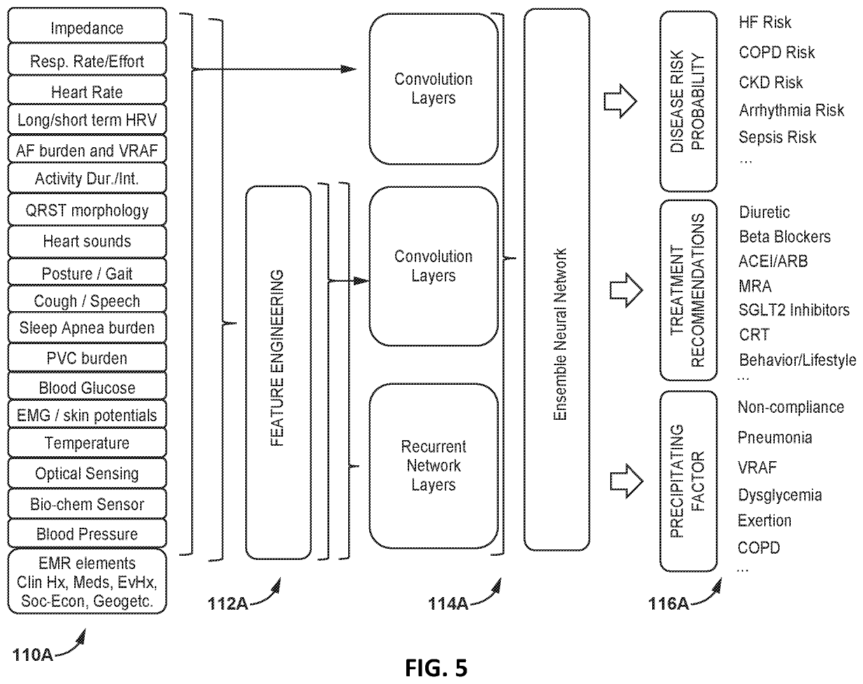

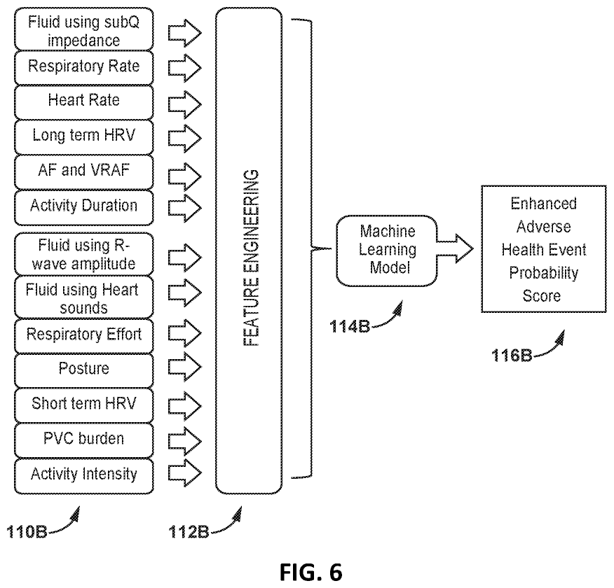

FIG. 5 illustrates an example of an AI model (which may include one or more ML models) that may be used as an example of a probability model as described herein. FIG. 6 illustrates an example of a ML model that may be used as an example of a probability model as described herein. As shown in FIGS. 5 and 6, values of a plurality of determined physiological parameters 110A and 110B, e.g., sensed or otherwise determined by medical device(s) 17 or other devices described herein, which may include raw or processed sensor signals, may be entered as inputs into probability models to determine a probability score indicating a likelihood that the patient is experiencing an adverse health event or is likely to experience the adverse health event within a predetermined amount of time. As illustrated in FIGS. 5 and 6, processing circuitry of system 2 may determine some or all of the input values for one or more ML models 114A and 114B based on feature engineering 112A and 112B, e.g., deriving input values from sensed physiological signals. FIG. 5 illustrates one or more ML models 114A including convolution layers and recurrent network layers as inputs to an ensemble neural network. However, any of a variety of ML or AI model architectures may be included.

Output 116B in FIG. 6 is a risk level, e.g., probability score, of an adverse health event, such as HF hospitalization or decompensation. Similarly, outputs 116A in FIG. 5 include risk levels for a variety of conditions discussed herein, such as HF, COPD, arrhythmia, sepsis, and chronic kidney disease (CKD). In some examples, as shown in FIG. 5, treatments, such as diuretics, beta blockers, behavior/lifestyle changes, ACE inhibitors, SGLT2 inhibitors, etc., may be recommended in response to the determined probability score indicating at least one of a likelihood that the patient (a) is experiencing an adverse health event or (b) is likely to experience the adverse health event. In some examples, as shown in FIG. 5, the determined probability score may also indicate a precipitating factor, such as COPD, exertion, dysglycemia, pneumonia, non-compliance, VRAF, etc., that led to the determined probability score. In some examples, the output 116A of one or more ML models 144A themselves may indicate one or more of diseases risk probability, treatment recommends, and/or precipitating factors.

In addition, medical device(s) 17 may also determine additional physiological parameter values such as a presence or absence or degree of panic attack, a hyperglycemic condition, a hypoglycemic condition, hypoxemia, anemia, an infection occurring during a period of time, such as at night that may increase a patient's likelihood of having a HF decompensation event. In some examples, medical device(s) 17 may include a continuous blood glucose monitor that includes various sensors to determine blood glucose of patient 4 to determine one or more of a hyperglycemic condition and a hypoglycemic condition. In some examples, medical device(s) 17 may include sensors to detect one or more of (or physiological parameters indicative of) a panic attack, anemia, and/or an infection in patient 4. In some examples, system 2 may determine physiological parameters, such as blood sugar, anemia, panic attack, and/or infection, based on measurements of sensors 62. In some examples, a user, such as a clinician or patient 4, may input information such as blood glucose data via external device 12 to indicate a hyperglycemic condition and/or a hypoglycemic condition. In some examples, a user, such as a clinician or patient 4, may input information via external device 12 to indicate a panic attack, anemia, and/or an infection in patient 4. In some examples, physiological parameters such as a panic attack, a hyperglycemic condition, a hypoglycemic condition, anemia, an infection may be received from electronic medical records stored in an external device.

In some examples, physiological parameters may be grouped as cardiac parameters, such as, but not limited to, impedance, heart rate, HRV, AF burden, VRAF, heart sounds, PVC burden, and QRST morphology. In some examples, physiological parameters may be grouped as non-cardiac parameters, such as, but not limited to, posture, activity, cough, speech, blood glucose, skin potentials, temperature, blood pressure, anemia, infection, hypoxemia, panic attack, etc. In some examples, physiological parameters may be grouped as glycemic parameters, such as blood glucose level, A1C level, occurrences of hypoglycemia, occurrences of hyperglycemia, etc. In some examples, physiological parameters may be grouped as breathing parameters, such as, but not limited to, respiratory rate, respiratory effort, sleep apnea burden, sleep disordered breathing.

System 2 may modify an algorithm for determining an adverse health event risk, such as HF, during periods of time when conditions such as a panic attack, a hyperglycemic condition, a hypoglycemic condition, anemia, an infection are also detected. In some examples, system 2 may combine multiple metrics, such as detection of a panic attack, a hyperglycemic condition, a hypoglycemic condition, anemia, an infection, using decision trees or fuzzy logic or a risk score using multivariate regression or deep learning neural networks techniques.

In some cases, data server(s) 94 may be configured to provide a secure storage site for data that has been collected from medical device(s) 17 and/or external device 12. In some instances, data server(s) 94 may include a database that stores medical- and health-related data. For example, data server(s) 94 may include a cloud server or other remote server that stores data collected from medical device(s) 17 and/or external device 12. In some cases, data server(s) 94 may assemble data in web pages or other documents for viewing by trained professionals, such as clinicians, via computing devices 100. One or more aspects of the example system described with reference to FIG. 1 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate medical device(s) 17. For example, the clinician may access data collected by medical device(s) 17 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by medical device(s) 17, external device 12, data server(s) 94, or any combination thereof, or based on other patient data known to the clinician.

One computing device 100 may transmit instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 (or relay an alert determined by a medical device 17, external device 12, or data sever 94) based on a probability score (e.g., posterior probability) determined from physiological parameter values of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 1, data server(s) 94 includes a storage device 96 (e.g., to store data retrieved from medical device(s) 17) and processing circuitry 98. Although not illustrated in FIG. 1 computing devices 100 may similarly include a storage device and processing circuitry. Processing circuitry 98 may include one or more processors that are configured to implement functionality and/or process instructions for execution within data server(s) 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of data server(s) 94 and/or the processing circuitry of computing devices 100 may implement any of the techniques described herein to analyze physiological parameters received from medical device(s) 17, e.g., to determine a probability score of patient 4.

In some examples, storage device 96 of data server(s) 94 may store and implement a probability model 19. In some examples, as illustrated in FIG. 2, external device 12 may store probability model 19. For example, data server(s) 94 may transmit probability model 19 to external device 12, where external device 12 may store the probability model 19 in a memory device of external device 12 (not shown in FIG. 1). External device 12 and/or data server(s) 94 may use the probability model 19 to determine a probability score with respect to a health risk for patient 4. One or more ML models 114A and 114B of FIGS. 5 and 6 may be examples of probability model 19.

Processing circuitry 98 of data server(s) 94 and/or the processing circuitry of computing devices 100 may also retrieve instructions for utilizing a selected probability model (e.g., one selected using a known selection technique) and execute the probability model to determine the probability score. Processing circuitry 98 of data server(s) 94 and/or the processing circuitry of computing devices 100 may retrieve such data and instructions from storage device 96 or in some instances, from another storage device, such as from one of medical devices 17.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

FIG. 2 is a block diagram illustrating an example configuration of components of external device 12. In some examples, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as one of medical device(s) 17 (e.g., IMD 10). Under the control of processing circuitry 80, communication circuitry 82 may receive downlink telemetry from, as well as send uplink telemetry to, one of medical device(s) 17 (e.g., IMD 10), or another device (e.g., data server(s) 94). Communication circuitry 82 may be configured to transmit or receive signals via inductive coupling, electromagnetic coupling, near-field communication (NFC) technologies (e.g., inductive coupling, NFC or other communication technologies operable at ranges less than 10-20 cm), RF communication, Bluetooth®, Wi-Fi™, or other proprietary or non-proprietary wireless communication schemes. Communication circuitry 82 may also be configured to communicate with devices other than medical device(s) 17 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Storage device 84 may store one or more probability models 19. Storage device 84 may also store historical data, diagnostic state data, physiological parameter values, probability scores, etc.

Data exchanged between external device 12 and medical device(s) 17 may include operational parameters (e.g., physiological parameter values, diagnostic states, etc.). External device 12 may transmit data including computer readable instructions which, when implemented by medical device(s) 17, may control medical device(s) 17 to change one or more operational parameters and/or export collected data (e.g., physiological parameter values). For example, processing circuitry 80 may transmit an instruction to medical device(s) 17 which requests medical device(s) 17 to export collected data (e.g., impedance data, fluid index values, and/or impedance scores, blood pressure, ECG records, any other parameters described above or elsewhere herein, etc.) to external device 12.

In turn, external device 12 may receive the collected data from medical device(s) 17 and store the collected data in storage device 84. Processing circuitry 80 may implement any of the techniques described herein to model physiological parameter values received from medical device(s) 17 to determine diagnostic states, probability scores, etc.

Using the modeling techniques disclosed herein, processing circuitry 80 may determine a likelihood that the patient is experiencing an adverse health event (e.g., heart failure decompensation) or is likely to experience an adverse health event within a predetermined amount of time (e.g., within the next 3 days, 7 days, 10 days, 30 days, 40 days, etc.). In an illustrative example, the predetermined amount of time may be at least approximately 7 days from when the probability score is determined, such that the probability score indicates the likelihood that an adverse health event will occur in the next 7 days or indicates that the patient is likely already experiencing an adverse health event, such as heart failure decompensation.

External device 12 may be a computing device with a display viewable by a user and an interface for providing input to external device 12 (i.e., a user input mechanism). The user may be a physician technician, surgeon, electrophysiologist, clinician, or patient 4. In some examples, external device 12 may be a notebook computer, tablet computer, computer workstation, one or more servers, cellular phone (smartphone), personal digital assistant, handheld computing device, networked computing device, or another computing device that may run an application that enables the computing device to interact with IMD 10. External device 12 is configured to communicate with IMD 10 and, optionally, another computing device, via wired or wireless communication. External device 12, for example, may communicate via NFC technologies (e.g., inductive coupling, NFC or other communication technologies operable at ranges less than 10-20 cm) and far-field communication technologies (e.g., Radio Frequency (RF) telemetry according to the 802.11 or Bluetooth® specification sets, or other communication technologies operable at ranges greater than NFC technologies). In some examples, external device 12 may include a programming head that may be placed proximate to the body of patient 4 near the IMD 10 implant site in order to improve the quality or security of communication between IMD 10 and external device 12.

In one example, a user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to medical device(s) 17 (e.g., cardiac ECGs, blood pressure, subcutaneous impedance values, RR, etc.). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

In some examples, user interface 86 of external device 12 may receive input from the user. The user interface may include, for example, a keypad and a display, which may for example, be a cathode ray tube (CRT) display, an LCD, or an LED display. The keypad may take the form of an alphanumeric keypad or a reduced set of keys associated with particular functions. External device 12 can additionally or alternatively include a peripheral pointing device, such as a mouse, via which the user may interact with the user interface. In some examples, a display of external device 12 may include a touch screen display, and a user may interact with external device 12 via the display. It should be noted that the user may also interact with external device 12 remotely via a networked computing device.

External device 12 may be coupled to external electrodes, or to implanted electrodes via percutaneous leads. In some examples, external device 12 may monitor impedance measurements from IMD 10. External device 12 may be used to configure operational parameters for IMD 10. For example, external device 12 may provide a parameter resolution for IMD 10 that indicates a resolution of data that IMD 10 should be obtaining. Examples of resolution parameters may include a frequency at which the electrodes process impedance measurements or a frequency at which impedance measurements should be considered in determining a diagnostic state.

In some examples, external device 12 may be one or more of a smartphone, a smartwatch, a wearable computing device, other smart apparel, personal computing devices, and IoT devices. IoT devices may include, as examples, so called “smart” speakers, cameras, televisions, lights, locks, thermostats, appliances, actuators, controllers, or any other smart home (or building) devices. IoT devices may provide audible and/or visual alarms when configured with output devices to do so. In some examples, IoT devices that include cameras or other sensors may activate those sensors to collect data regarding patient 4, e.g., for evaluation of the condition of patient 4. In some examples, external device 12 may be any one or more computing device configured for wireless communication with medical device(s) 17, such as a desktop, laptop, or tablet computer. External device 12 may communicate with medical device(s) 17 according to the Bluetooth® or Bluetooth® Low Energy (BLE) protocols, as examples.

Power source 108 delivers operating power to the components of external device 12. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within external device 12. In other embodiments, traditional batteries (e.g., nickel cadmium or lithium ion batteries) may be used. In addition, external device 12 may be directly coupled to an alternating current outlet to power external device 12. Power source 108 may include circuitry to monitor power remaining within a battery. In this manner, user interface 86 may provide a current battery level indicator or low battery level indicator when the battery needs to be replaced or recharged. In some cases, power source 108 may be capable of estimating the remaining time of operation using the current battery.

FIG. 7 illustrates the environment of an example medical system 2 in conjunction with a patient 4, in accordance with one or more techniques of this disclosure. Patient 4 ordinarily, but not necessarily, will be a human. For example, patient 4 may be an animal needing ongoing monitoring for cardiac conditions.

In some examples, system 2 may include IMD 10. In other examples, system 2 may not include IMD 10 and may instead include other medical device(s) 17 (not shown in FIG. 7). IMD 10 may include one or more electrodes (not shown) on its housing, or may be coupled to one or more leads that carry one or more electrodes. System 2 may also include external device 12 and, although not depicted in FIG. 7, the various other devices illustrated in one or more of the various example techniques described with reference to FIG. 1. Example system 2 may be used to measure physiological parameters to provide to patient 4 or other user a probability score indicating a likelihood that the patient is experiencing an adverse health event or is likely to experience the adverse health event within a predetermined amount of time.

The example techniques may be used with an IMD 10, which may be in wireless communication with at least one of external device 12 or data server(s) 94. In some examples, IMD 10 is implanted outside of a thoracic cavity of patient 4 (e.g., subcutaneously in the pectoral location illustrated in FIG. 7). IMD 10 may be positioned near the sternum near or just below the level of the heart of patient 4, e.g., at least partially within the cardiac silhouette. IMD 10 may include a plurality of electrodes and may be configured for subcutaneous implantation outside of a thorax of patient 4.

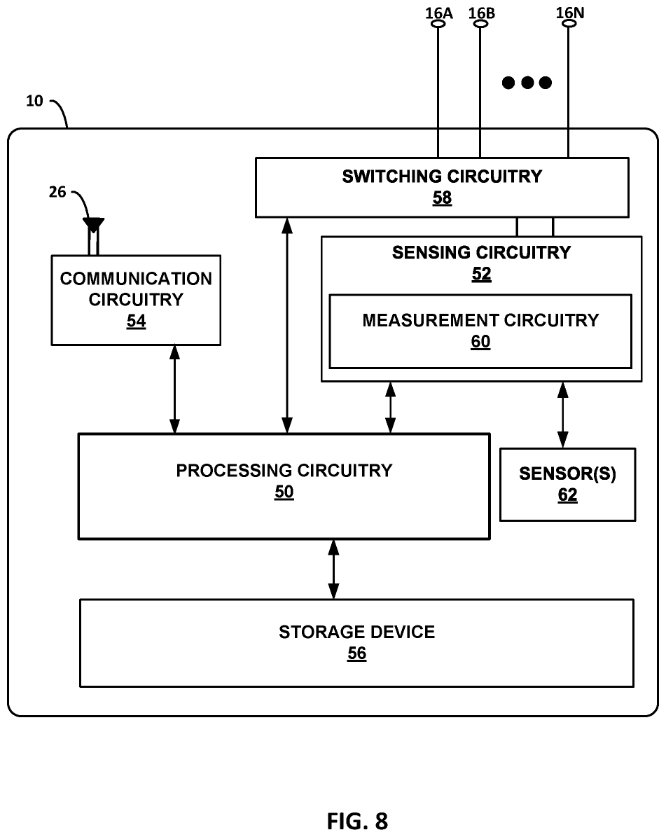

Accordingly, impedance measurements may be taken via electrodes in the subcutaneous space, e.g., electrodes on a subcutaneously implanted medical device as shown in FIGS. 7-8, may be measurements of the impedance of interstitial fluid and subcutaneous tissue. Implantable medical devices (IMDs) can sense and monitor impedance signals and use those signals to determine a health condition status of a patient or other health condition status of a patient (e.g., edema, preeclampsia, hypertension, etc.). The electrodes used by IMDs to sense impedance signals are typically integrated with a housing of the IMD and/or coupled to the IMD via one or more elongated leads. Example IMDs that include electrodes include the Reveal LINQ™ or LINQ II™ Insertable Cardiac Monitor (ICM), developed by Medtronic, Inc., of Minneapolis, MN, which may be inserted subcutaneously. Other example IMDs may include electrodes on a subcutaneous lead connected to another one of medical device(s) 17, such as a subcutaneous implantable cardioverter-defibrillator (ICD) or an extravascular ICD. Such IMDs may facilitate relatively longer-term monitoring of patients during normal daily activities and may periodically transmit collected data to a network service, such as the Medtronic CareLink® Network.

System 2 may measure subcutaneous impedance of patient 4 and processes impedance data to accumulate evidence of decreasing impedance. The accumulated evidence is referred to as a fluid index and may be determined as function of the difference between measured impedance values and reference impedance values. The fluid index may then be used to determine impedance scores that are indicative of a heart condition of patient 4.

In some examples, IMD 10 may also sense cardiac electrogram (ECG) signals via the plurality of electrodes and/or operate as a therapy delivery device. For example, IMD 10 may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

In some examples, system 2 may include any suitable number of leads coupled to IMD 10, and each of the leads may extend to any location within or proximate to a heart or in the chest of patient 4.

Although described in the context of examples in which IMD 10 includes an insertable or implantable IMD, example systems including one or more external devices of any type configured to sense impedance parameters and other physiological parameters may be configured to implement the techniques of this disclosure. In some examples, IMD 10 may be a device configured to measure impedances of a fluid and shifts in impedances of the fluid, such as interstitial fluid. For example, IMD 10 may have one or more electrodes disposed within one layer of patient 4 (e.g., subcutaneous layer), whereas at least one other electrode may be disposed within another layer of patient 4 (e.g., dermis layer, muscle layer, etc.). In such examples, IMD 10 may be able to measure impedances and shifts in impedances of the interstitial fluid of the subcutaneous layer. In another example, IMD 10 may be a cutaneous patch device having electrodes on the outside of the skin. In such examples, IMD 10 may use the cutaneous patch device to measure impedances and shifts in impedances of the interstitial fluid in the subcutaneous layer.

In examples in which IMD 10 also operates as a pacemaker, a cardioverter, and/or defibrillator, or otherwise monitors the electrical activity of the heart, IMD 10 may sense electrical signals attendant to the depolarization and repolarization of the heart of patient 4 via electrodes of or coupled to IMD 10, e.g., which may include the electrodes used to determine subcutaneous impedance and other physiological parameters. In some examples, IMD 10 can provide pacing pulses to the heart of patient 4 based on the electrical signals sensed within the heart of patient 4. The configurations of electrodes used by IMD 10 for sensing and pacing may be unipolar or bipolar. IMD 10 may also provide defibrillation therapy and/or cardioversion therapy via electrodes located on at least one lead, as well as a housing electrode. IMD 10 may detect tachyarrhythmia of the heart of patient 4, such as fibrillation of atria or ventricles, and deliver defibrillation or other tachyarrhythmia therapy to the heart of patient 4 in the form of electrical pulses. In some examples, IMD 10 may be programmed to deliver a progression of therapies, e.g., pulses with increasing energy levels, until a fibrillation of the heart of patient 4 is stopped. IMD 10 detects fibrillation or other tachyarrhythmias employing tachyarrhythmia detection techniques known in the art.

FIG. 8 is a functional block diagram illustrating an example configuration of IMD 10. IMD 10 may include an example of one of medical device(s) 17 described with reference to FIGS. 2-4. In the illustrated example, IMD 10 includes electrodes 16A-16N (collectively, “electrodes 16”), antenna 26, processing circuitry 50, sensing circuitry 52, measurement circuitry 60, communication circuitry 54, storage device 56, switching circuitry 58, sensors 62. IMD 10, along with other medical device(s) 17, may also include a power source. In general, the power source may include a rechargeable or non-rechargeable battery. Each of medical device(s) 17 may include components common to those of IMD 10. For example, each of medical device(s) 17 may include processing circuitry 50. For sake of brevity, each configuration of each medical device(s) 17 will not be described in this application. That is, certain components of IMD 10 may serve as representative components of other medical device(s) 17 (e.g., storage device 56, communication circuitry 54, sensor(s) 62, etc.).

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense impedance and/or cardiac signals, as controlled by processing circuitry 50. Sensing circuitry 52 may sense signals from electrodes 16, e.g., to produce a cardiac ECG, in order to facilitate monitoring the electrical activity of the heart. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

In some examples, processing circuitry 50 may use switching circuitry 58 to select, e.g., via a data/address bus, which of the available electrodes are to be used to obtain impedance measurements of interstitial fluid and to sense cardiac signals, and to select the polarities of the electrodes. Switching circuitry 58 may include a switch array, switch matrix, multiplexer, transistor array, microelectromechanical switches, or any other type of switching device suitable to selectively couple sensing circuitry 58 to selected electrodes. In some examples, sensing circuitry 52 includes one or more sensing channels, each of which may include an amplifier. In response to the signals from processing circuitry 50, switching circuitry 58 may couple the outputs from the selected electrodes to one of the sensing channels.

In some examples, one or more channels of sensing circuitry 52 may include one or more R-wave amplifiers that receive signals from electrodes 16. In some examples, the R-wave amplifiers may take the form of an automatic gain-controlled amplifier that provides an adjustable sensing threshold as a function of the measured R-wave amplitude. In addition, in some examples, one or more channels of sensing circuitry 52 may include a P-wave amplifier that receives signals from electrodes 16. Sensing circuitry may use the received signals for pacing and sensing in the heart of patient 4. In some examples, the P-wave amplifier may take the form of an automatic gain-controlled amplifier that provides an adjustable sensing threshold as a function of the measured P-wave amplitude. Other amplifiers may also be used.

In some examples, processing circuitry 50 may be configured to record an R-wave amplitude for an ECG sensed by sensing circuitry 52. For example, sensing circuitry 52 may be configured to sense a subcutaneous ECG, and processing circuitry 50 may be configured to record an R-wave amplitude of the subcutaneous ECG. In another example, sensing circuitry 52 may be configured to record cardiac electrogram using leads in the heart of patient 4 and as measured between the housing of one of medical device(s) 17 (e.g., a can) and the leads in the heart of patient 4, and processing circuitry 50 may be configured to record an R-wave amplitude of the cardiac electrogram. Similarly, sensing processing circuitry 50 may record R-wave slopes or R-wave widths for an ECG or other cardiac electrogram.

In some examples, sensing circuitry 52 includes a channel that includes an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in storage device 56. In some examples, processing circuitry 50 may employ digital signal analysis techniques to characterize the digitized signals stored in storage device 56 to detect P-waves (e.g., within ventricular or far-field signals and instead of or in addition to use of P-wave amplifiers) and classify cardiac tachyarrhythmias from the digitized electrical signals.

Based on the detection of R-waves and P-waves, e.g., their rates, processing circuitry 50 may identify atrial and ventricular tachyarrhythmias, such as AF or VF. Processing circuitry may employ digital signal analysis techniques to detect or confirm such tachyarrhythmias in some examples. Processing circuitry 50 may determine values of physiological parameters based on detection of such tachyarrhythmias, and a probability of a health event may be determined based on the physiological parameter values according to the techniques described herein. Example physiological parameters determined based on detection of tachyarrhythmia include an extent, e.g., frequency and/or duration during a time period, of AF or other tachyarrhythmias.

Processing circuitry 50 may also determine other physiological parameter values that can be used to determine probability of a health event based on the cardiac ECG and detection of depolarizations therein. As examples, processing circuitry 50 may determine one or more heart rate values, such as night heart rate values, one or more heart rate variability values. As other examples, processing circuitry 50 may determine magnitudes of or intervals between features within the cardiac ECG, such as depolarization amplitudes, depolarization widths, or intervals between depolarizations and repolarizations.

In some examples, sensors 62 include one or more accelerometers or other sensors configured to generate signals that indicate motion and orientation of patient 4, e.g., that indicate activity level or posture of the patient. In some examples, processing circuitry 50 processes such signals to determine values of one or more physiological parameters that can be used to determine probability of a health event. For example, processing circuitry 50 may quantify duration, frequency, and/or intensity of activity and/or posture changes, e.g., daily or during some other period. In some examples, processing circuitry 50 may determine an amount of time patient 4 spends inactive, e.g., sleeping, but not in a supine posture based on such signals.

Sensing circuitry 52 may include measurement circuitry 60. Processing circuitry 50 may control measurement circuitry 60 to periodically measure an electrical parameter to determine an impedance, such as a subcutaneous impedance indicative of fluid found in interstitium.

In some examples, processing circuitry 50 may perform physiological parameter measurement, such as an impedance measurement, by causing measurement circuitry 60 (via switching circuitry 58) to deliver a voltage pulse between at least two electrodes 16 and examining resulting current amplitude value measured by measurement circuitry 60. In some examples, switching circuitry 58 delivers signals that do deliver stimulation therapy to the heart of patient 4. In other examples, these signals may be delivered during a refractory period, in which case they may not stimulate the heart of patient 4.

In some examples, processing circuitry 50 may perform an impedance measurement in accordance with techniques described in the applications incorporated by reference, as discussed above. In some examples, IMD 10 may use an amplifier circuit according to certain techniques described in U.S. application Ser. No. 12/872,552 by Denison et al., entitled “CHOPPER-STABILIZED INSTRUMENTATION AMPLIFIER FOR IMPEDANCE MEASUREMENT,” filed on Aug. 31, 2010, incorporated herein by reference in its entirety, to for physiological signal sensing, impedance sensing, telemetry, etc.

Sensing circuitry 52 may also provide one or more physiological parameter signals to processing circuitry 50 for analysis, e.g., for analysis to determine physiological parameters, e.g., posture angle, short term HRV. IMD 10 may include one or more sensors 62, such as one or more accelerometers, gyroscopes, microphones, optical sensors, temperature sensors, pressure sensors, and/or chemical sensors. In some examples, processing circuitry 50 may store the physiological parameter values, score physiological parameter factors (e.g., posture angle, average heart rate, activity, etc.), and scores in storage device 56. Processing circuitry 50 of IMD 10, and/or processing circuitry of another device that retrieves data from IMD 10, may analyze the values to determine a diagnostic state of the respective physiological parameter.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network.

Antenna 26 and communication circuitry 54 may be configured to transmit and/or receive signals via inductive coupling, electromagnetic coupling, NFC technologies, RF communication, Bluetooth®, Wi-Fi™, or other proprietary or non-proprietary wireless communication schemes. In some examples, processing circuitry 50 may provide data to be uplinked to external device 12 via communication circuitry 54 and control signals using an address/data bus. In another example, communication circuitry 54 may provide received data to processing circuitry 50 via a multiplexer.

In some examples, processing circuitry 50 may send physiological parameter data to external device 12 or data server(s) 94 via communication circuitry 54. For example, IMD 10 may send external device 12 or data server(s) 94 collected physiological parameter measurements. External device 12 and/or data server(s) 94 may then analyze those physiological parameter measurements.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media. For example, storage device 56 may include random access memory (RAM), read-only memory (ROM), non-volatile RAM (NVRAM), electrically erasable programmable ROM (EEPROM), erasable programmable ROM (EPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include physiological parameter values and/or digitized cardiac ECGs, as examples.

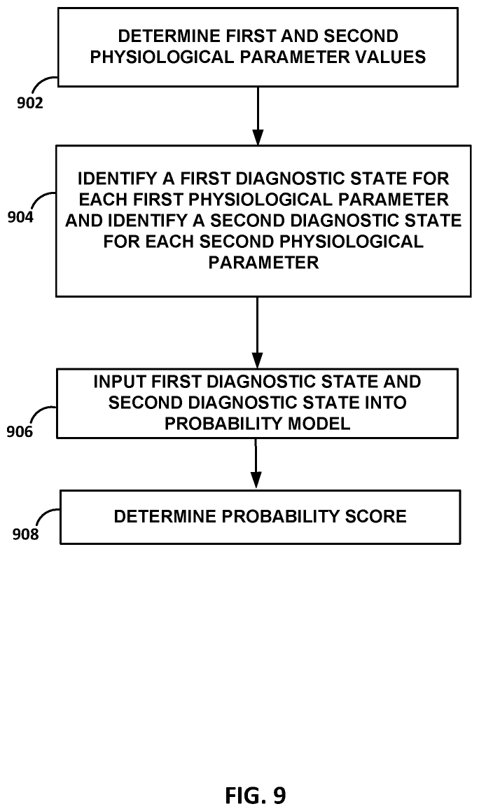

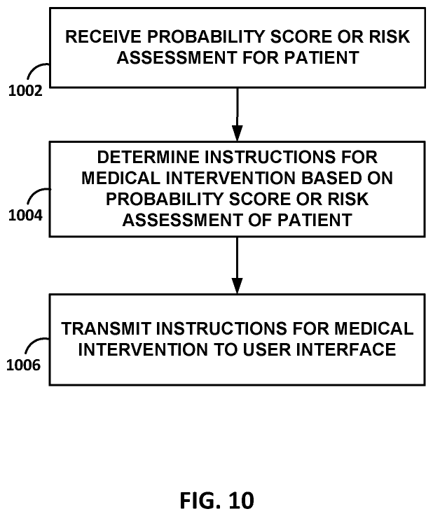

FIGS. 9 and 10 illustrate example methods that may be performed by one or more of medical devices 17, external device 12, and/or data server(s) 94 in conjunction with probability model 19 described herein to determine a probability score with respect to the patient, in accordance with one or more techniques disclosed herein. Although described as being performed by data server(s) 94, one or more of the various example techniques described with reference to FIGS. 9 and 10 may be performed by any one or more of IMD 10, external device 12, or data server(s) 94, e.g., by the processing circuitry of any one or more of these devices.

In some examples, processing circuitry, e.g., processing circuitry 50 of IMD 10, processing circuitry 80 of external device 12, or processing circuitry 98 of data server(s) 94, may determine values of first physiological parameters and second physiological parameters, such as those first and second physiological parameters described herein (902). For each of the first physiological parameters and each of the second physiological parameters, processing circuitry 98 may identify diagnostic states (904). In some instances, processing circuitry 98 may filter irrelevant physiological parameters or parameter values prior to identifying diagnostic states or after identifying diagnostic states. For example, certain physiological parameters or values thereof may not be relevant to the purpose of deploying probability model 19. In such instances, those physiological parameters or values may still be available to processing circuitry 80, but processing circuitry 98 may determine those parameters should not be used as inputs to the probability model. At any point in time, processing circuitry 98 may access probability model 19. For example, processing circuitry 98 may access probability model 19 stored in storage device 96.

In some examples, processing circuitry 98 may determine conditional likelihood and prior probability data. For example, processing circuitry 98 may determine conditional likelihood and prior probability data in accordance with the techniques described in U.S. application Ser. No. 13/391,376 by Sarkar et al., entitled “METHOD AND APPARATUS FOR MONITORING TISSUE FLUID CONTENT FOR USE IN AN IMPLANTABLE CARDIAC DEVICE,” filed on Feb. 20, 2012, and incorporated herein by reference in its entirety. For example, the conditional likelihood parameters may take the form of conditional likelihood tables defined for each first diagnostic state for each first physiological parameter and for each second diagnostic state for each second physiological parameter. The posterior probability may then be tabulated for all possible combinations of diagnostic states to determine a posterior probability, or in some instances, a probability table, as described in U.S. application Ser. No. 13/391,376.

Processing circuitry 98 may then input the first diagnostic state(s) and the second diagnostic state(s) into the probability model 19 (906). Processing circuitry 98 may execute probability model 19 in response to a user command or may do so automatically in response to a triggering event. For example, processing circuitry 98 may determine that all necessary diagnostic states have been determined and that the probability model 19 is ready for execution to determine a probability score. In some examples, processing circuitry 98 may receive data for various physiological parameters or processing circuitry 98 may access data from storage device 96. Processing circuitry 98 may determine the diagnostic state (L/M/H) for each parameter. For the particular set of first physiological parameters and second physiological parameters used (e.g., parameters having sufficient data), processing circuitry 98 may use the diagnostic states to map to a particular row of the LUT. Processing circuitry 98 may identify the posterior probability that is mapped to the particular row. Processing circuitry 98 may subsequently utilize the data that was used to determine the posterior probability to train the probability model 19 for future rounds of determining probability scores. For example, processing circuitry 98 may use the current or incoming data to determine the prior probability value(s).

In some examples, processing circuitry 98 may execute probability model a number of times in a finite period of time in order to determine an average probability score for the finite period of time. In some instances, processing circuitry 98 may execute probability model in accordance with a Monte Carlo simulation (e.g., using repeated random sampling to obtain numerical results).

As such, processing circuitry 98 may determine one or more probability scores from probability model 19 (908). Processing circuitry 98 may use the first diagnostic state and the second diagnostic state as inputs to probability model 19 to determine a posterior probability score that indicates a likelihood that a patient will experience an adverse health event within a predetermine period of time (e.g., within 30 days of determining the probability score). As discussed herein, the probability model may use the prior probability value and a conditional likelihood parameter as additional inputs to determine the posterior probability score. The probability score (p-value) may be represented as a decimal value. In some examples, processing circuitry may then update the probability model using the determined posterior probability score. In some examples, a Bayesian ML model may determine the probability score, where the ML model may be trained on prior probability values and feedback received regarding the accuracy of the probability score in predicting adverse health events. In some examples, a BBN model may determine the probability score, where the BBN model may be trained on prior probability values and feedback received regarding the accuracy of the probability score in predicting adverse health events. In some examples, a deep learning model may determine the probability score, where the deep learning model may be trained on prior probability values and feedback received regarding the accuracy of the probability score in predicting adverse health events.

Processing circuitry 98 may compare the probability score to at least one risk threshold. Based on the comparison, processing circuitry 98 may determine one of plurality of discrete risk categorizations. For example, discrete risk categorizations may be high risk, low risk, or medium risk. Although described as being performed by server 94, one or more of the various example techniques described with reference to FIG. 9 may be performed by any one or more of IMD 10, external device 12, or server 94, e.g., by the processing circuitry of any one or more of these devices. In this way, processing circuitry 98 may determine a health risk status for a patient based at least in part on the probability score. In an example, processing circuitry 98 may be configured to monitor a patient, for example, with heart failure for a developing heart failure decompensation in accordance with one or more of the various techniques disclosed herein to then allow for early intervention potentially before clinical symptoms are experienced or hospitalization is needed. In another example, through implementation of the disclosed probability model (e.g., a Bayesian model), processing circuitry 98 may combine information from multiple “orthogonal” parameters, that alone may not be very specific, to ultimately gain a more specific classification.

In some examples, where the first measurements include heart rate measurements and the second measurements patient activity measurements, processing circuitry 98 may collate the heart rate measurements and activity measurements over a period of time. Processing circuitry 98 may apply a line fit to the collated measurements to determine a rate of change of heart rate as a function of change in activity over the period time, determine a slope of the applied line fit, compare the determined slope to a threshold, determine the second diagnostic state based on the comparison, and may determine the second diagnostic state is chronotropic incompetence that indicates an increased the likelihood the patient is experiencing the adverse health event or is likely to experience the adverse health event within the predetermined amount of time.

In some examples, where the second measurements include posture angle measurements and patient activity measurements, processing circuitry 98 may determine a first period of time when the patient is active based on the activity measurements and determine a second period of time when the patient is at rest based on the activity measurements. Processing circuitry 98 may collate the plurality of posture angle measurements during the first period of time, collate the plurality of posture angle measurements during the second period of time, determine a first posture angle during based on the plurality of posture angle measurements during the first period of time. determine a second posture angle based on the plurality of posture angle measurements during the second period of time, compare the determined first posture angle to the determined second posture angle, determine the second diagnostic state based on the comparison, and may determine the second diagnostic state is a low posture difference that indicates an increased the likelihood the patient is experiencing the adverse health event or is likely to experience the adverse health event within the predetermined amount of time.

In some examples, where the second measurements include short term HRV measurements, processing circuitry 98 may determine HRV metrics based on the HRV measurements and collate the determined HRV metrics over a period of time. Processing circuitry 98 may compare collated HRV metrics to a respective threshold, determine the second diagnostic state based on the comparison, and determine the second diagnostic state is a high mode-sum value that indicates an increased the likelihood the patient is experiencing the adverse health event or is likely to experience the adverse health event within the predetermined amount of time. In some examples, the determined second diagnostic state may be two states, such as a high risk or a low risk. In some examples, the determined second diagnostic state may include a plurality of states, such as receiving a risk score, such as, but not limited to, between 0-100, that indicates the risk level of the determined second diagnostic state.

In some examples, where the subcutaneous tissue impedance measurements include interstitial impedance measurements, and the second measurements include patient activity measurements, processing circuitry 98 may collate the measured interstitial impedance over a first period of time. Processing circuitry 98 may determine a second period of time when the patient is inactive based on the activity measurements, the second period of time being within the first period of time, determine the second diagnostic state based on the measured interstitial impedances over the second period of time, may determine the second diagnostic state is sleep disordered breathing that indicates an increased the likelihood the patient is experiencing the adverse health event or is likely to experience the adverse health event within the predetermined amount of time.

In some examples, where the first measurements include an ECG signal, processing circuitry 98 may collate R-wave features based on R-waves of the ECG signal. Processing circuitry 98 compare collated R-wave features to a respective threshold, determine the second diagnostic state based on the comparison, and may determine the second diagnostic state is one or more of decreasing R-wave amplitude and increasing QRS complex duration that indicates an increased the likelihood the patient is experiencing the adverse health event or is likely to experience the adverse health event within the predetermined amount of time.

It should be noted that one or more of the various example techniques described with reference to FIG. 9 may be performed on a periodic basis. For example, the probability score may be determined according to a resolution parameter setting of medical device(s) 17 and/or for patient 4. In other examples, the probability score may be calculated irrespective of the resolution parameter. Data server(s) 94 may calculate the probability score once a day, each week, every two weeks, each month, etc. In some examples, data server(s) 94 may also calculate the probability score in response to a user command (e.g., from a physician, from a user interface) or in response to a satisfaction of another condition, such as upon receiving or determining a particular number of diagnostic states, or an indication of a change in the condition of patient from a source other than the application of the probability model.

Data server(s) 94 may also trigger a probability score determination when an activity level or other physiological parameter of patient 4 satisfies a threshold (e.g., low activity when patient 4 is resting or sleeping). In one other example, data server(s) 94 or medical device 17, e.g., IMD 10, may determine the probability score of patient 4 on a per measurement basis, such as on a per impedance score determination basis. A person of skill in the art would appreciate that various periods may exist for when IMD 10 or external device 12 may determine a probability score in accordance with FIG. 9, transmit a probability score, etc. In some examples, data server(s) 94 may determine the probability score of patient 4 in response to receipt of physiological parameter data for patient via network 92, e.g., from medical device(s) 17.

In addition, although described in terms of data server(s) 94 performing one or more of the various example techniques of this disclosure, it is to be understood that any number of different components described with reference to, for example, FIGS. 4, 5, 7, and 8, and combinations thereof, may perform one or more of the various example techniques of this disclosure. For example, IMD 10 or external device 12 may determine diagnostic states 11 or physiological parameter values. In addition, data server(s) 94 may include multiple computing devices (e.g., a remote cloud server) that collectively determines probability scores of patient 4 experiencing an adverse health event within a predetermined period of time. In addition, it is to be understood that the example components described with reference to FIGS. 4, 5, 7, and 8 may perform some or all of the example techniques described with reference to FIGS. 4, 5, and 10 in parallel or in conjunction with one another.

According to the example of FIG. 10, data server(s) 94 may determine or receive the probability score or risk assessment for patient 4 from probability model 19 (1002). In some examples, the probability score may include a discrete risk categorization. For example, processing circuitry 98 may compare the probability score to at least one risk threshold. In some examples, processing circuitry 98 may perform such comparisons on a daily basis, weekly basis, monthly basis, etc. with or without real-time alerts and/or notifications. The risk thresholds may include discrete risk categorizations, such as above 20%=high risk, above 5%=medium risk and below 5%=low risk. Thus, processing circuitry 98 may determine, based on the comparison of the probability score to at least one risk threshold, a discrete risk categorization (e.g., high risk) from a plurality of discrete risk categorizations. As such, a risk assessment includes either the probability score or the risk categorization based on a risk threshold determination.

In some examples, data server(s) 94 may receive the probability score or risk assessment of patient 4 (1002). In some examples, data server(s) 94 may determine the probability score (>20%) or risk assessment (high risk). In some examples, a threshold to determine whether the probability score is considered high may be user programmable and/or may be based on patient population. Data server(s) 94 may determine instructions for medical intervention based on the probability score or risk assessment of patient 4 (1004). For example, if the probability score is greater than a high-risk threshold, data server(s) 94 may determine instructions for medical intervention based on the high-risk determination. In other examples, data server(s) 94 may determine different instructions for different risk levels or categories. For example, data server(s) 94 may determine a first set of instructions for a high-risk patient and a second set of instructions for a medium-risk patient. In some examples, data server(s) 94 may not determine any instructions for a low risk patient (e.g., probability score less than 20%). In some examples, data server(s) 94 may generate an alert notification or sound an audible or tactile alarm alerting of the high-risk determination. In one example, the alert may include text or graphics information that communicates the probability score to an interested party. In addition, data server(s) 94 may provide information regarding the risk determination, such as a summary or detailed report of the alert. In a non-limiting example, the information may state that processing circuitry 98 determined high fluid based on impedance scores, high RR, and new onset AF, but that NHR and HRV was indicated as being normal, with activity being in the OK range. In some examples, external device 12 may provide a visual light indication, such as emitting a red light for high risk or a yellow light for medium risk. The alert may indicate a possible or predicted heart failure decompensation event that is likely to occur within a predetermined period of time.

In general, one cause of HF hospitalization (HFH) involves volume overload in which the body of a patient retains an excess amount of fluid. In such instances, the primary HF management strategy is to control excess fluid volume using diuretic and/or vasodilator or nitrate therapy. Further, ACE-Inhibitors, which control blood pressure, and B-blockers, which control heart rate, may reduce mortality in HF patients. As such, posterior probability may indicate how much therapy should be administered. For example, a medical device 17 may be configured to deliver a therapy and/or data server(s) 94 may be configured to provide a therapy instruction based on a posterior probability that satisfies a particular risk threshold. Posterior probability may include a percentage (e.g., 20%) or a decimal value (0.2) or a probability score (e.g., high, medium, low; intervention, light intervention, no intervention; etc.) determined from the percentage or decimal value. In some examples, posterior probability may include a probability distribution, such as a Gaussian distribution, where processing circuitry 98 may determine a likelihood percentage, decimal value, or probability score, from the probability distribution.

In some examples, data server(s) 94 may transmit the instructions for medical intervention to a user interface (1006). In other examples, data server(s) 94 may transmit the instructions to a device of a caretaker, such as a pager. In examples where IMD 10 generates the instructions based on a probability score, processing circuitry 50 may transmit the instructions for medical intervention to a user interface. The instructions may include the probability score or may include the diagnostic states that factored into determining the probability score. In some instances, a physician or caretaker may not need to know the diagnostic states and may only want to receive the probability score determined from the diagnostic states or vice versa. In any event, processing circuitry may compare the probability score against at least one risk threshold on a periodic or semi-periodic basis. In some examples, medical intervention techniques may be assessed or processing circuitry, e.g., processing circuitry 50 of IMD 10, processing circuitry 80 of external device 12, or processing circuitry 98 of data server(s) 94, may provide adjustments to patient treatment in accordance with certain techniques described in commonly-assigned U.S. application Ser. No. 15/402,839 by Sharma et al., entitled “MEDICAL SYSTEM FOR SEAMLESS THERAPY ADJUSTMENT,” filed on Jan. 10, 2017, incorporated herein by reference in its entirety.

Various examples have been described. However, one of ordinary skill in the art will appreciate that various modifications may be made to the described examples without departing from the scope of the claims. For example, additional physiological parameters may be considered to determine probability scores of worsening heart failure or other adverse health events. Examples of other physiological parameters are described in commonly-assigned U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, both of which are incorporated herein by reference in their entirety.

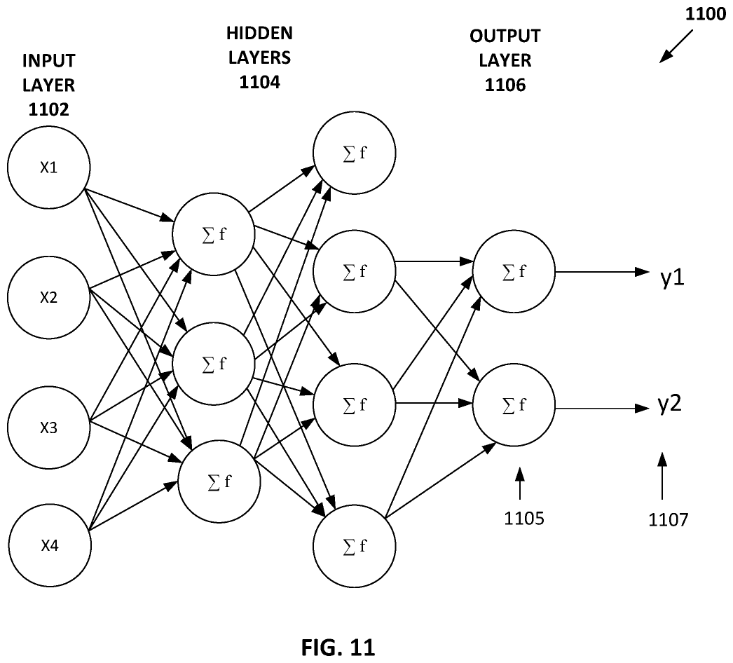

FIG. 11 is a conceptual diagram illustrating an example ML model 1100 configured to generate one or more values indicative of a risk of a health event, e.g., for heart failure or another patient condition, based on physiological parameter values, e.g., sensed by an IMD and/or other devices as described herein. ML model 1100 may be an example of probability model 19 and of ML models 114 in FIGS. 5 and 6. ML model 1100 is an example of a deep learning model, or deep learning algorithm. One or more of IMD 10, external device 12, or sever 94 may train, store, and/or utilize machine learning model 1100, but other devices may apply inputs associated with a particular patient to machine learning model 1100 in other examples. Some non-limiting examples of machine learning techniques include Bayesian probability models, Support Vector Machines, K-Nearest Neighbor algorithms, and Multi-layer Perceptron.

As shown in the example of FIG. 11, machine learning model 1100 may include three layers. These three layers include input layer 1102, hidden layer 1104, and output layer 1106. Output layer 1106 comprises the output from the transfer function 1105 of output layer 1106. Input layer 1102 represents each of the input values X1 through X4 provided to machine learning model 1100. The number of inputs may be less than or greater than 4, including much greater than 4, e.g., hundreds or thousands. In some examples, the input values may be any of the of physiological or other patient parameter values described herein.

Each of the input values for each node in the input layer 1102 is provided to each node of hidden layer 1104. In the example of FIG. 11, hidden layers 1104 include two layers, one layer having four nodes and the other layer having three nodes, but fewer or greater number of nodes may be used in other examples. Each input from input layer 1102 is multiplied by a weight and then summed at each node of hidden layers 1104. During training of machine learning model 1100, the weights for each input are adjusted to establish the relationship between input physiological parameter values and one or more output values indicative of a risk level of a health event for the patient. In some examples, one hidden layer may be incorporated into machine learning model 1100, or three or more hidden layers may be incorporated into machine learning model 1100, where each layer includes the same or different number of nodes.

The result of each node within hidden layers 1104 is applied to the transfer function of output layer 1106. The transfer function may be liner or non-linear, depending on the number of layers within machine learning model 1100. Example non-linear transfer functions may be a sigmoid function or a rectifier function. The output 1107 of the transfer function may be a value or values indicative of a risk of an HF event or other health event of the patient. By applying the patient parameter data to a machine learning model, such as machine learning model 1100, processing circuitry of system 2 is able to determine the health condition with great accuracy, specificity, and sensitivity.

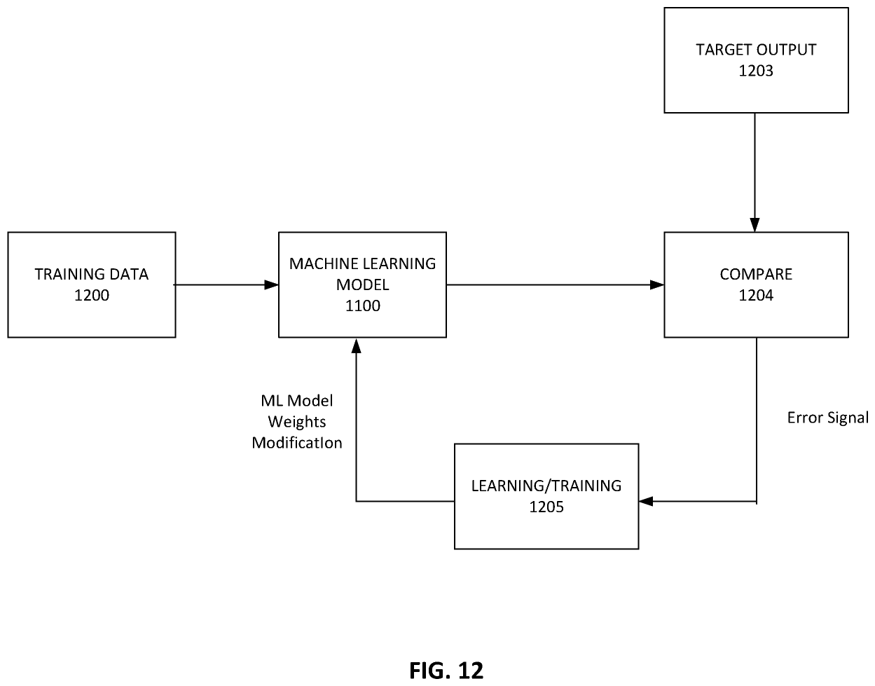

FIG. 12 is an example of a machine learning model 1100 being trained using supervised and/or reinforcement learning techniques. Machine learning model 1100 may be implemented using any number of models for supervised and/or reinforcement learning, such as but not limited to, an artificial neural network, a decision tree, naïve Bayes network, support vector machine, or k-nearest neighbor model, to name only a few examples. In some examples, processing circuitry one or more of IMD 10, external device 12, and/or server 94 initially trains the machine learning model 1100 based on training set data 1200 including numerous instances of input data corresponding to various risk levels of a health event. An output of the machine learning model 1100 may be compared 1204 to the target output 1203, e.g., as determined based on the label. Based on an error signal representing the comparison, the processing circuitry implementing a learning/training function 1205 may send or apply a modification to weights of machine learning model 1100 or otherwise modify/update the machine learning model 1100. For example, one or more of IMD 10, external device 12, and/or server 94 may, for each training instance in the training set 1200, modify machine learning model 1100 to change a score generated by the machine learning model 1100 in response to data applied to the machine learning model 1100.

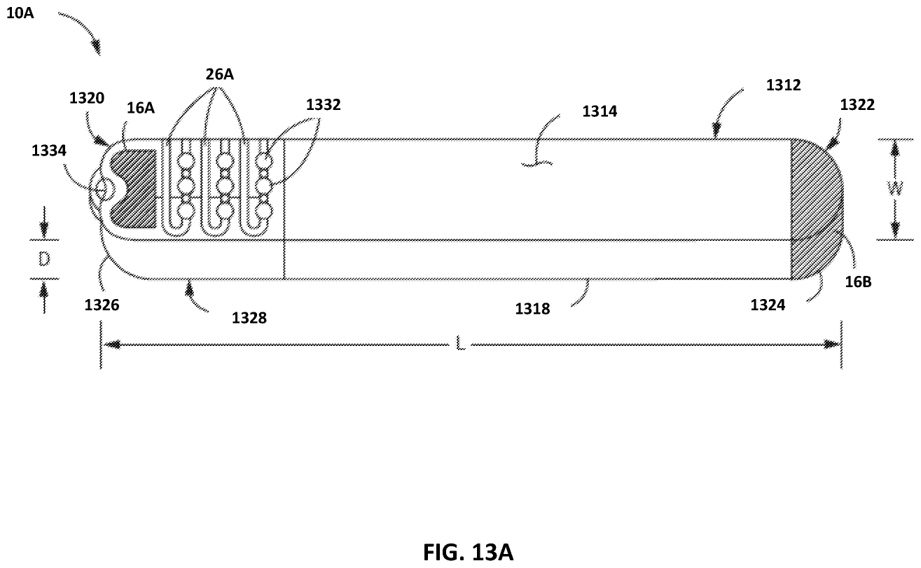

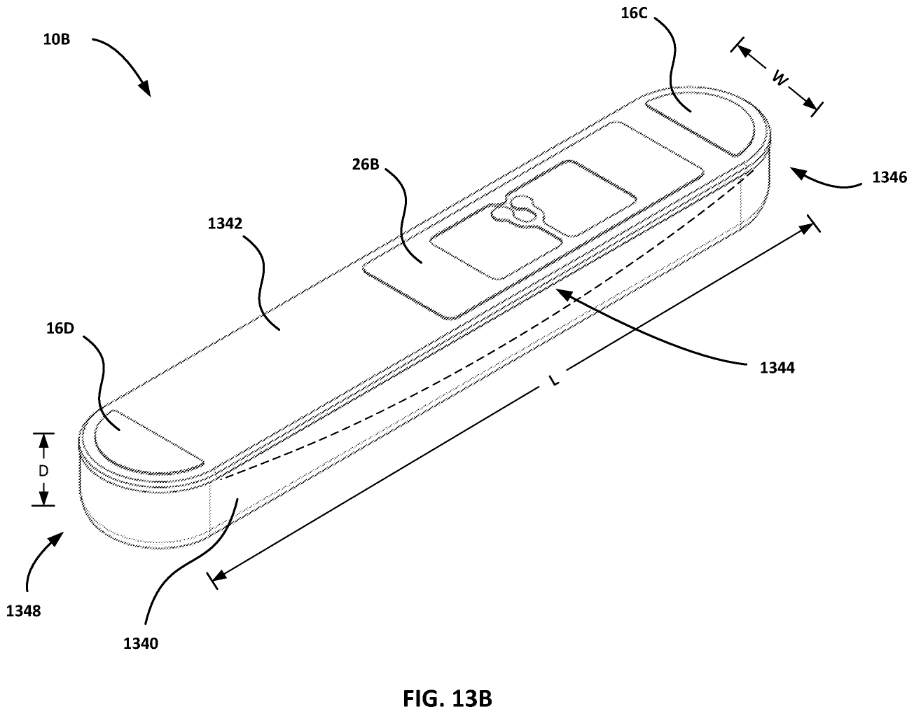

FIG. 13A is a perspective drawing illustrating an IMD 10A, which may be an example configuration of IMD 10 of FIGS. 7 and 8 as an ICM. In the example shown in FIG. 13A, IMD 10A may be embodied as a monitoring device having housing 1312, proximal electrode 16A and distal electrode 16B. Housing 1312 may further comprise first major surface 1314, second major surface 1318, proximal end 1320, and distal end 1322. Housing 1312 encloses electronic circuitry located inside the IMD 10A and protects the circuitry contained therein from body fluids. Housing 1312 may be hermetically scaled and configured for subcutaneous implantation. Electrical feedthroughs provide electrical connection of electrodes 16A and 16B.

In the example shown in FIG. 13A, IMD 10A is defined by a length L, a width W and thickness or depth D and is in the form of an elongated rectangular prism wherein the length L is much larger than the width W, which in turn is larger than the depth D. In one example, the geometry of the IMD 10A—in particular a width W greater than the depth D—is selected to allow IMD 10A to be inserted under the skin of the patient using a minimally invasive procedure and to remain in the desired orientation during insertion. For example, the device shown in FIG. 10A includes radial asymmetries (notably, the rectangular shape) along the longitudinal axis that maintains the device in the proper orientation following insertion. For example, the spacing between proximal electrode 16A and distal electrode 16B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 1314 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for case of implant and patient comfort. Examples of IMD 10A described in this disclosure may have a volume of three cubic centimeters (cm) or less, 1.5 cubic cm or less or any volume between three and 1.5 cubic cm.

In the example shown in FIG. 13A, once inserted within the patient, the first major surface 1314 faces outward, toward the skin of the patient while the second major surface 1318 is located opposite the first major surface 1314. In addition, in the example shown in FIG. 13A, proximal end 1320 and distal end 1322 are rounded to reduce discomfort and irritation to surrounding tissue once inserted under the skin of the patient. IMD 10A, including instrument and method for inserting IMD 10A is described, for example, in U.S. Patent Publication No. 2014/0276928, incorporated herein by reference in its entirety.

Proximal electrode 16A is at or proximate to proximal end 1320, and distal electrode 16B is at or proximate to distal end 1322. Proximal electrode 16A and distal electrode 16B are used to sense cardiac EGM signals, e.g., ECG signals, and measure interstitial impedance thoracically outside the ribcage, which may be sub-muscularly or subcutaneously. EGM signals and impedance measurements may be stored in a memory of IMD 10A, and data may be transmitted via integrated antenna 26A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, from any implanted location. Housing 1312 may house the circuitry of IMD 10 illustrated in FIG. 8.

In the example shown in FIG. 10A, proximal electrode 16A is at or in close proximity to the proximal end 1320 and distal electrode 16B is at or in close proximity to distal end 1322. In this example, distal electrode 16B is not limited to a flattened, outward facing surface, but may extend from first major surface 1314 around rounded edges 1324 and/or end surface 1326 and onto the second major surface 1318 so that the electrode 16B has a three-dimensional curved configuration. In some examples, electrode 16B is an uninsulated portion of a metallic, e.g., titanium, part of housing 1312.

In the example shown in FIG. 13A, proximal electrode 16A is located on first major surface 1314 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 1314 similar to that shown with respect to proximal electrode 16A.

The various electrode configurations allow for configurations in which proximal electrode 16A and distal electrode 16B are located on both first major surface 1314 and second major surface 1318. In other configurations, such as that shown in FIG. 13A, only one of proximal electrode 16A and distal electrode 16B is located on both major surfaces 1314 and 1318, and in still other configurations both proximal electrode 16A and distal electrode 16B are located on one of the first major surface 1314 or the second major surface 1318 (e.g., proximal electrode 16A located on first major surface 1314 while distal electrode 16B is located on second major surface 1318). In another example, IMD 10A may include electrodes on both major surface 1314 and 1318 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 13A, proximal end 1320 includes a header assembly 1328 that includes one or more of proximal electrode 16A, integrated antenna 26A, anti-migration projections 1332, and/or suture hole 1334. Integrated antenna 26A is located on the same major surface (i.e., first major surface 1314) as proximal electrode 16A and is also included as part of header assembly 1328. Integrated antenna 26A allows IMD 10A to transmit and/or receive data. In other examples, integrated antenna 26A may be formed on the opposite major surface as proximal electrode 16A, or may be incorporated within the housing 1312 of IMD 10A. In the example shown in FIG. 10A, anti-migration projections 1332 are located adjacent to integrated antenna 26A and protrude away from first major surface 1314 to prevent longitudinal movement of the device. In the example shown in FIG. 13A, anti-migration projections 1332 include a plurality (e.g., nine) small bumps or protrusions extending away from first major surface 1314. As discussed above, in other examples anti-migration projections 1332 may be located on the opposite major surface as proximal electrode 16A and/or integrated antenna 26A. In addition, in the example shown in FIG. 13A, header assembly 1328 includes suture hole 1334, which provides another means of securing IMD 10A to the patient to prevent movement following insertion. In the example shown, suture hole 1334 is located adjacent to proximal electrode 16A. In one example, header assembly 1328 is a molded header assembly made from a polymeric or plastic material, which may be integrated or separable from the main portion of IMD 10A.

FIG. 13B is a perspective drawing illustrating another IMD 10B, which may be another example configuration of IMD 10 from FIGS. 7 and 8 as an ICM. IMD 10B of FIG. 13B may be configured substantially similarly to IMD 10A of FIG. 13A, with differences between them discussed herein.

IMD 10B may include a leadless, subcutaneously-implantable monitoring device, e.g. an ICM. IMD 10B includes housing having a base 1340 and an insulative cover 1342. Proximal electrode 16C and distal electrode 16D may be formed or placed on an outer surface of cover 1342. Various circuitries and components of IMD 10B, e.g., described with respect to FIG. 8, may be formed or placed on an inner surface of cover 1342, or within base 1340. In some examples, a battery or other power source of IMD 10B may be included within base 1340. In the illustrated example, antenna 26B is formed or placed on the outer surface of cover 1342, but may be formed or placed on the inner surface in some examples. In some examples, insulative cover 1342 may be positioned over an open base 1340 such that base 1340 and cover 1342 enclose the circuitries and other components and protect them from fluids such as body fluids. The housing including base 1340 and insulative cover 1342 may be hermetically sealed and configured for subcutaneous implantation.

Circuitries and components may be formed on the inner side of insulative cover 1342, such as by using flip-chip technology. Insulative cover 1342 may be flipped onto a base 1340. When flipped and placed onto base 1340, the components of IMD 10B formed on the inner side of insulative cover 1342 may be positioned in a gap 1344 defined by base 1340. Electrodes 16C and 16D and antenna 26B may be electrically connected to circuitry formed on the inner side of insulative cover 1342 through one or more vias (not shown) formed through insulative cover 1342. Insulative cover 1342 may be formed of sapphire (i.e., corundum), glass, parylene, and/or any other suitable insulating material. Base 1340 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 13B, the housing of IMD 1310B defines a length L, a width W and thickness or depth D and is in the form of an elongated rectangular prism wherein the length L is much larger than the width W, which in turn is larger than the depth D, similar to IMD 10A of FIG. 13A. For example, the spacing between proximal electrode 16C and distal electrode 16D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 13B, once inserted subcutaneously within the patient, outer surface of cover 1342 faces outward, toward the skin of the patient. In addition, as shown in FIG. 13B, proximal end 1346 and distal end 1348 are rounded to reduce discomfort and irritation to surrounding tissue once inserted under the skin of the patient. In addition, edges of IMD 10B may be rounded.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: A system includes an implantable medical device (IMD) includes obtain second measurements, the second measurements being different than the first measurements; determine a first respective one or more values for each of a plurality of first physiological parameters, wherein each of the plurality of first physiological parameters indicates a physiological status of the patient, the plurality of first physiological parameters determined from the one or more first measurements; determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from the one or more second measurements; identify a first diagnostic state for each of the first physiological parameters based on the first respective values and identify a second diagnostic state for each of the second physiological parameters based on the second respective values, the first and second diagnostic states defining a plurality of inputs for a probability model; and determine, from the probability model, a probability score indicating at least one of a likelihood that the patient (a) is experiencing an adverse health event of a chronic condition of the patient or (b) is to experience the adverse health event.

Example 2: The system of example 1, wherein the system further includes a second device external to the patient to measure the second measurements.

Example 3: The system of example 2, wherein the second device includes one or more of: smartphone, scale, bed sensor, biochemical sensor, camera, smart device, wearable computing device, continuous glucose monitor.

Example 4: The system of any of examples 1 through 3, wherein the processing circuitry is configured to determine, from the probability model, a probability score indicating a likelihood that the patient is likely to experience the adverse health event within a predetermined amount of time.

Example 5: The system of any of examples 1 through 4, wherein the physiological status of the patient includes one or more of: heart rate, respiratory rate, fluid status, sympathetic tone, heart rate variability (HRV), blood pressure, fluid redistribution, tissue perfusion, pulse oxygenation, or sleep disordered breathing.

Example 6: The system of any of examples 1 through 5, wherein one or more of the second physiological parameters indicates the precipitating condition of the patient, and the precipitating condition includes one or more of: current clinical status, clinical history, weight, pneumonia, sepsis, respiratory infection, chronic obstructive pulmonary disease (COPD), atrial fibrillation with rapid ventricular rate, anemia, hypoxemia, hyperglycemia, hypoglycemia, panic attack, physical exertions, dietary non-compliance, medication non-compliance, dietary change, medication change, reduction in urinary output, sleep apnea, Cheyenes Stokes breathing, sleep apnea burden, premature ventricular contractions (PVC) burden, or increased fluid consumption.

Example 7: The system of any of examples 1 through 6, wherein one or more of the second physiological parameters indicates the symptom of the patient, and the symptom of the patient includes one or more of respiratory rate, respiratory effort, rales through lung sounds, symptom app, coughing, cough frequency, chronotropic incompetence, hematocrit, and peripheral perfusion of incident infection.

Example 8: The system of any of examples 1 through 7, wherein one or more of the second physiological parameters indicates the functional capacity of the patient, and the functional capacity of the patient includes one or more of: activity, voice pattern, sleep posture, gait, and speech pattern.

Example 9: The system of any of examples 1 through 8, wherein the first measurements include heart rate measurements, and the second measurements patient activity measurements, and the processing circuitry is further configured to: collate the heart rate measurements and activity measurements over a period of time; apply a line fit to the collated measurements to determine a rate of change of heart rate as a function of change in activity over the period time; determine a slope of the applied line fit; compare the determined slope to a threshold; and determine the second diagnostic state based on the comparison.

Example 10: The system of example 9, wherein the determined second diagnostic state is chronotropic incompetence to indicate an increased the likelihood the patient (a) is experiencing the adverse health event or (b) is to experience the adverse health event.

Example 11: The system of any of examples 1 through 10, wherein the second measurements include posture angle measurements and patient activity measurements, and the processing circuitry is further configured to: determine a first period of time when the patient is active based on the activity measurements; determine a second period of time when the patient is at rest based on the activity measurements; collate the plurality of posture angle measurements during the first period of time; collate the plurality of posture angle measurements during the second period of time; determine a first posture angle during based on the plurality of posture angle measurements during the first period of time; determine a second posture angle based on the plurality of posture angle measurements during the second period of time; compare the determined first posture angle to the determined second posture angle; and determine the second diagnostic state based on the comparison.

Example 12: The system of example 11, wherein the determined second diagnostic state is a low posture difference to indicate an increased the likelihood the patient (a) is experiencing the adverse health event or (b) is to experience the adverse health event.

Example 13: The system of any of examples 1 through 12, wherein the second measurements include short-term heart rate variability (HRV) measurements, and the processing circuitry is further configured to: determine HRV metrics based on the HRV measurements; collate the determined HRV metrics over a period of time; compare collated HRV metrics to a respective threshold; and determine the second diagnostic state based on the comparison.

Example 14: The system of example 13, wherein the determined second diagnostic state is a high mode-sum value to indicate an increased the likelihood the patient (a) is experiencing the adverse health event or (b) is to experience the adverse health event.

Example 15: The system of any of examples 1 through 14, wherein the first measurements include interstitial impedance measurements, and the second measurements include patient activity measurements, and the processing circuitry is further configured to: collate the measured interstitial impedance over a first period of time; determine a second period of time when the patient is inactive based on the activity measurements, the second period of time being within the first period of time; and determine the second diagnostic state based on the measured interstitial impedances over the second period of time.

Example 16: The system of example 15, wherein the determined second diagnostic state is sleep disordered breathing to indicate an increased the likelihood the patient (a) is experiencing the adverse health event or (b) is to experience the adverse health event.

Example 17: The system of any of examples 1 through 16, wherein the first measurements include an electrocardiogram (ECG) signal, and the processing circuitry is further configured to: collate R-wave features based on R-waves of the ECG signal; compare collated R-wave features to a respective threshold; and determine the second diagnostic state based on the comparison.

Example 18: The system of example 17, wherein the determined second diagnostic state is one or more of decreasing R-wave amplitude, increasing QRS complex duration, and increasing QRS width to indicate an increased the likelihood the patient (a) is experiencing the adverse health event or (b) is to experience the adverse health event.

Example 19: A method includes determining a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements; determining a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements; identifying a first diagnostic state for each of the first physiological parameters based on the first respective values; identifying a second diagnostic state for each of the second physiological parameters based on the second respective values, the first and second diagnostic states defining a plurality of inputs for a probability model; and determining, from the probability model, a probability score indicating at least one of a likelihood that the patient (a) is experiencing an adverse health event of a chronic condition of the patient or (b) is to experience the adverse health event.

Example 20: The method of example 19, further includes determining, from the probability model, a probability score indicating a likelihood that the patient is likely to experience the adverse health event within a predetermined amount of time.

Example 21: The method of any of examples 19 and 20, wherein the first measurements include heart rate measurements, and the second measurements include patient activity measurements, the method further includes collating the heart rate measurements and activity measurements over a period of time; applying a line fit to the collated measurements to determine a rate of change of heart rate as a function of change in activity over the period time; determining a slope of the applied line fit; comparing the determined slope to a threshold; and determining the second diagnostic state based on the comparison.

Example 22: The method of any of examples 19 through 21, wherein the second measurements include posture angle measurements and patient activity measurements, the method further includes determining a first period of time when the patient is active based on the activity measurements; determining a second period of time when the patient is at rest based on the activity measurements; collating the plurality of posture angle measurements during the first period of time; collating the plurality of posture angle measurements during the second period of time; determining a first posture angle during based on the plurality of posture angle measurements during the first period of time; determining a second posture angle based on the plurality of posture angle measurements during the second period of time; comparing the determined first posture angle to the determined second posture angle; and determining the second diagnostic state based on the comparison.

Example 23: The method of any of examples 19 through 22, wherein the second measurements include short-term heart rate variability (HRV) measurements, the method further includes determining HRV metrics based on the HRV measurements; collating the determined HRV metrics over a period of time; comparing collated HRV metrics to a respective threshold; and determining the second diagnostic state based on the comparison.

Example 24: The method of any of examples 19 through 23, wherein the first measurements include interstitial impedance measurements, and the second measurements include patient activity measurements, the method further includes collating the measured interstitial impedance over a first period of time; determining a second period of time when the patient is inactive based on the activity measurements, the second period of time being within the first period of time; and determining the second diagnostic state based on the measured interstitial impedances over the second period of time.

Example 25: The method of any of examples 19 through 24, wherein the first measurements include an electrocardiogram (ECG) signal, the method further includes collating R-wave features based on r-waves of the ECG signal; comparing collated R-wave features to a respective threshold; and determining the second diagnostic state based on the comparison.

Example 26: A non-transitory computer-readable storage medium having stored thereon instructions that, when executed, cause one or more processors to at least: determine a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements; determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements; identify a first diagnostic state for each of the first physiological parameters based on the first respective values; identify a second diagnostic state for each of the second physiological parameters based on the second respective values, the first and second diagnostic states defining a plurality of inputs for a probability model; and determine, from the probability model, a probability score indicating at least one of a likelihood that the patient (a) is experiencing an adverse health event of a chronic condition of the patient or (b) is to experience the adverse health event.

Various examples have been described. These and other examples are within the scope of the following claims.

1. A system comprising:
an implantable medical device (IMD) comprising a plurality of electrodes and configured for subcutaneous implantation in a patient, wherein the IMD is configured to determine one or more first measurements comprising subcutaneous tissue impedance measurements via the electrodes; and
processing circuitry coupled to one or more storage devices, and configured to:
obtain second measurements, the second measurements being different than the first measurements;
determine a first respective one or more values for each of a plurality of first physiological parameters, wherein each of the plurality of first physiological parameters indicates a physiological status of the patient, the plurality of first physiological parameters determined from the one or more first measurements;
determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from the one or more second measurements;
identify a first diagnostic state for each of the first physiological parameters based on the first respective values and identify a second diagnostic state for each of the second physiological parameters based on the second respective values, the first and second diagnostic states defining a plurality of inputs for a probability model; and
determine, from the probability model, a probability score indicating at least one of a likelihood that the patient (a) is experiencing an adverse health event of a chronic condition of the patient or (b) is to experience the adverse health event.

2. The system of claim 1, wherein the system further includes a second device external to the patient to measure the second measurements.

3. The system of claim 2, wherein the second device includes one or more of: smartphone, scale, bed sensor, biochemical sensor, camera, smart device, wearable computing device, continuous glucose monitor.

4. The system of claim 1, wherein the processing circuitry is configured to determine, from the probability model, a probability score indicating a likelihood that the patient is likely to experience the adverse health event within a predetermined amount of time.

5. The system of claim 1, wherein the physiological status of the patient includes one or more of: heart rate, respiratory rate, fluid status, sympathetic tone, heart rate variability (HRV), blood pressure, fluid redistribution, tissue perfusion, pulse oxygenation, or sleep disordered breathing.

6. The system of claim 1, wherein one or more of the second physiological parameters indicates the precipitating condition of the patient, and the precipitating condition includes one or more of: current clinical status, clinical history, weight, pneumonia, sepsis, respiratory infection, chronic obstructive pulmonary disease (COPD), atrial fibrillation with rapid ventricular rate, anemia, hypoxemia, hyperglycemia, hypoglycemia, panic attack, physical exertions, dietary non-compliance, medication non-compliance, dietary change, medication change, reduction in urinary output, sleep apnea, Cheyenes Stokes breathing, sleep apnea burden, premature ventricular contractions (PVC) burden, or increased fluid consumption.

7. The system of claim 1, wherein one or more of the second physiological parameters indicates the symptom of the patient, and the symptom of the patient includes one or more of respiratory rate, respiratory effort, rales through lung sounds, symptom app, coughing, cough frequency, chronotropic incompetence, hematocrit, and peripheral perfusion of incident infection.

8. The system of claim 1, wherein one or more of the second physiological parameters indicates the functional capacity of the patient, and the functional capacity of the patient includes one or more of: activity, voice pattern, sleep posture, gait, speech pattern, and sit-to-stand time.

9. The system of claim 1, wherein the first measurements include heart rate measurements, and the second measurements patient activity measurements, and the processing circuitry is further configured to:
collate the heart rate measurements and activity measurements over a period of time;
apply a line fit to the collated measurements to determine a rate of change of heart rate as a function of change in activity over the period time;
determine a slope of the applied line fit;
compare the determined slope to a threshold; and
determine the second diagnostic state based on the comparison.

10. The system of claim 9, wherein the determined second diagnostic state is chronotropic incompetence to indicate an increased the likelihood the patient (a) is experiencing the adverse health event or (b) is to experience the adverse health event.

11. The system of claim 1, wherein the second measurements include posture angle measurements and patient activity measurements, and the processing circuitry is further configured to:
determine a first period of time when the patient is active based on the activity measurements;
determine a second period of time when the patient is at rest based on the activity measurements;
collate the plurality of posture angle measurements during the first period of time;
collate the plurality of posture angle measurements during the second period of time;
determine a first posture angle during based on the plurality of posture angle measurements during the first period of time;
determine a second posture angle based on the plurality of posture angle measurements during the second period of time;
compare the determined first posture angle to the determined second posture angle; and
determine the second diagnostic state based on the comparison.

12. The system of claim 11, wherein the determined second diagnostic state is a low posture difference to indicate an increased the likelihood the patient (a) is experiencing the adverse health event or (b) is to experience the adverse health event.

13. The system of claim 1, wherein the second measurements include short-term heart rate variability (HRV) measurements, and the processing circuitry is further configured to:
determine HRV metrics based on the HRV measurements;
collate the determined HRV metrics over a period of time;
compare collated HRV metrics to a respective threshold; and
determine the second diagnostic state based on the comparison.

14. The system of claim 13, wherein the determined second diagnostic state is a high mode-sum value to indicate an increased the likelihood the patient (a) is experiencing the adverse health event or (b) is to experience the adverse health event.

15. The system of claim 1, wherein
the first measurements include interstitial impedance measurements, and the second measurements include patient activity measurements, and
the processing circuitry is further configured to:
collate the measured interstitial impedance over a first period of time;
determine a second period of time when the patient is inactive based on the activity measurements, the second period of time being within the first period of time; and
determine the second diagnostic state based on the measured interstitial impedances over the second period of time.

16. The system of claim 15, wherein the determined second diagnostic state is sleep disordered breathing to indicate an increased the likelihood the patient (a) is experiencing the adverse health event or (b) is to experience the adverse health event.

17. The system of claim 1, wherein the first measurements include an electrocardiogram (ECG) signal, and the processing circuitry is further configured to:
collate R-wave features based on R-waves of the ECG signal;
compare collated R-wave features to a respective threshold; and
determine the second diagnostic state based on the comparison.

18. The system of claim 17, wherein the determined second diagnostic state is one or more of decreasing R-wave amplitude, increasing QRS complex duration, and increasing QRS width to indicate an increased the likelihood the patient (a) is experiencing the adverse health event or (b) is to experience the adverse health event.

19. The system of claim 1, wherein the implantable medical device comprises an insertable cardiac monitor comprising:
a housing configured for subcutaneous implantation in the patient, the housing having a length between 40 millimeters (mm) and 60 mm between a first end and a second end, a width less than the length, and a depth less than the width;
a first electrode of the plurality of electrodes at or proximate to the first end; and
a second electrode of the plurality of electrodes at or proximate to the second end,
wherein the insertable cardiac monitor is configured to determine the subcutaneous tissue impedance measurements via the first electrode and the second electrode.

20. The system of claim 1, further comprising one or more computing devices configured to communicate with the implantable medical device, wherein the one or more computing devices comprise the processing circuitry.

21. A method comprising:
determining a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements;
determining a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements;
identifying a first diagnostic state for each of the first physiological parameters based on the first respective values;
identifying a second diagnostic state for each of the second physiological parameters based on the second respective values, the first and second diagnostic states defining a plurality of inputs for a probability model; and
determining, from the probability model, a probability score indicating at least one of a likelihood that the patient (a) is experiencing an adverse health event of a chronic condition of the patient or (b) is to experience the adverse health event.

22. A non-transitory computer-readable storage medium having stored thereon instructions that, when executed, cause one or more processors to at least:
determine a first respective one or more values for each of a plurality of first physiological parameters, wherein each of the plurality of first physiological parameters indicates a physiological status of the patient, the plurality of first physiological parameters being determined from one or more first measurements comprising subcutaneous tissue impedance measurements;
determine a second respective one or more values for each of a plurality of second physiological parameters, wherein each of the plurality of second physiological parameters indicates at least one of a precipitating condition of the patient, a symptom of the patient, or a functional capacity of the patient, the plurality of second physiological parameters determined from one or more second measurements, the second measurements being different than the first measurements;
identify a first diagnostic state for each of the first physiological parameters based on the first respective values;
identify a second diagnostic state for each of the second physiological parameters based on the second respective values, the first and second diagnostic states defining a plurality of inputs for a probability model; and
determine, from the probability model, a probability score indicating at least one of a likelihood that the patient (a) is experiencing an adverse health event of a chronic condition of the patient or (b) is to experience the adverse health event.
